(19) United States
(12) Patent Application Publication
(10) Pub. No.: US 2014/0288323 A1
(43) Pub. Date: Sep. 25, 2014

(54) CYCLOPROPENIMINE CATALYST COMPOSITIONS AND PROCESSES

(71) Applicant: THE TRUSTEES OF COLUMBIA UNIVERSITY IN THE CITY OF NEW YORK, New York, NY (US)

(72) Inventors: Tristan Hayes Lambert, Monroe, NY (US); Jeffrey Bandar, New York, NY (US)

(73) Assignee: The Trustees of Columbia University in the City of New York, New York, NY (US)

(21) Appl. No.: 14/352,526

(22) PCT Filed: Oct. 15, 2012
(86) PCT No.: PCT/US12/60243

§ 371 Date: Apr. 17, 2014

Related U.S. Application Data
(60) Provisional application No. 61/549,066, filed on Oct. 19, 2011.
(60) Provisional application No. 61/606,972, filed on Mar. 5, 2012.

(51) Int. Cl. C07C 249/02; B01J 31/02

(52) U.S. Cl. CPC C07C 249/02; B01J 31/0241; B01J 31/0271
USPC 560/38; 564/248

(57) ABSTRACT

The present invention provides, inter alia, a cyclopropenimine Brønsted base catalyst and a cyclopropenimine scaffold for use as a Brønsted base catalyst. This cyclopropenimine has the structure (100). Methods for making such a cyclopropenimine are also provided. Further provided are processes for carrying out an organic synthetic reaction and processes for catalyzing a proton transfer reaction enantioselectively using such a cyclopropenimine Brønsted base catalyst.

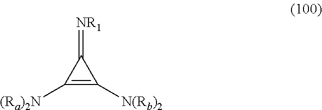

### CROSS-REFERENCE TO RELATED APPLICATIONS

The present invention claims benefit to U.S. provisional application Ser. No. 61/549,066, filed Oct. 19, 2011 and No. 61/606,972 filed Mar. 5, 2012. The entire contents of the above applications are incorporated by reference.

### GOVERNMENT FUNDING

This invention was made with government support under grant no. CHE-0953259 from the National Science Foundation. The government has certain rights in the invention.

### FIELD OF INVENTION

The present invention provides, inter alia, cyclopropenimine Brønsted base catalysts. Processes for making and using such cyclopropenimine Brønsted base catalysts are also provided.

### BACKGROUND OF THE INVENTION

Due to the prevalence of chemical reactions involving proton transfer as a key mechanistic event, Brønsted bases have become indispensable tools for the practice of organic synthetic chemistry (Ishikawa, 2009). Of particular interest in recent years has been the development of chiral Brønsted bases capable of catalyzing proton transfer reactions enantioselectively for the production of optically enriched products (Palomo et al., 2009; Marcelli et al., 2010, Leow et al., 2010; Fu et al., 2011). Although enantioselective Brønsted base catalysis holds great promise, this area has arguably lagged far behind development of other modes of asymmetric catalysis.

In general, a Brønsted base catalyst must possess a strength of basicity properly tuned to the acidity of a given substrate. In this regard, strong, neutral organic bases such as DBU (diazabicycloundecene) or TMG (tetramethylguanidine) have proven highly useful as reagents or catalysts for numerous transformations (Palomo et al., 2009; Marcelli et al., 2010, Leow et al., 2010; Fu et al., 2011). However, the amidine and guanidine functionalities upon which these and related reagents are built have inherent limitations of basicity, which has inhibited the development of broadly effective chiral catalysts based on these structures. Significantly stronger basicities can be realized with phosphazene (Schwesinger et al., 1987) or phosphatrane (Milbrath et al., 1977) structures, and a number of these reagents have become important additions to the Brønsted base arsenal. Nevertheless, broadly effective chiral catalysts based on these functionalities have not yet been realized.

### SUMMARY OF THE INVENTION

In view of the foregoing, there exists a strong need for novel Brønsted bases that provide potent yet tunable basicity, are trivial to prepare, and offer unique opportunities for asymmetric transition state organization. This invention is directed to meeting these and other needs.

Accordingly, one embodiment of the present invention is a cyclopropenimine Brønsted base catalyst. This catalyst has the structure:

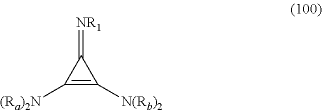

wherein

R₁ is independently selected from the group consisting of aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀cycloalkenyl, C₂₋₁₀alkynyl, halogen, aryloxy, heteroaryloxy, C₂₋₁₀alkoxycarbonyl, C₁₋₁₀alkylthio, C₂₋₁₀alkenylthio, C₂₋₁₀alkynylthio, C₁₋₁₀alkylsulfonyl, C₁₋₁₀alkylsulfinyl aryl-C₁₋₁₀alkyl, heteroaryl-C₁₋₁₀alkyl, aryl-C₁₋₁₀heteroalkyl, heteroaryl-C₁₋₁₀heteroalkyl; wherein the aliphatic or aromatic portions of R₁ are optionally substituted with from 1 to 4 substituents selected from the group consisting of halogen, cyano, nitro, C₁₋₄-alkyl, C₂₋₆alkenyl, C₂₋₆alkynyl, aryl, C₁₋₆alkoxy, C₂₋₆alkenyloxy, C₂₋₆alkynyloxy, aryloxy, C₂₋₆alkoxycarbonyl, C₁₋₆ alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus groups; and

each Rₐ and R_{b} are the same or different and are independently selected from sterically demanding substituents; and

crystalline forms, hydrates, or salts thereof.

Another embodiment of the present invention is a cyclopropenimine scaffold for use as a Brønsted base catalyst, which forms a cyclopropenium ion upon protonation of an imino nitrogen. This scaffold has the structure:

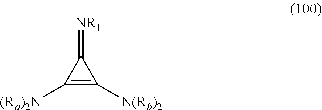

wherein

R₁ is a hydroxyl containing substituent; and

each Rₐ and R_{b} are the same or different and are independently selected from sterically demanding substituents; and

crystalline forms, hydrates, or salts thereof.

Yet another embodiment of the present invention is a process for making a cyclopropenimine suitable for use as a Brønsted base catalyst. This process comprises contacting a compound of formula (110) with an amine under conditions suitable to form a cyclopropenimine Brønsted base catalyst of formula (100):

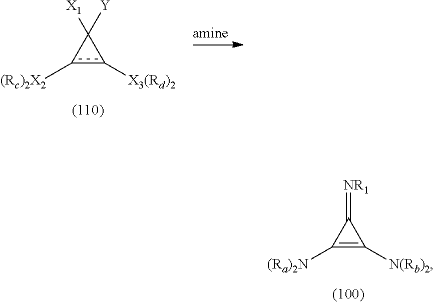

wherein
each X₁₋₃ and Y are the same or different and are independently selected from the group consisting of Cl, N, and no atom;
each Rₐ, R_{b}, R_{c} and R_{d} are the same or different and are independently selected from the group consisting of no atom, amino, aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀cycloalkenyl, C₂₋₁₀alkynyl, halogen, aryloxy, heteroaryloxy, C₂₋₁₀alkoxycarbonyl, C₁₋₁₀alkylthio, C₂₋₁₀alkenylthio, C₂₋₁₀alkynylthio, C₁₋₁₀alkylsulfonyl, C₁₋₁₀alkylsulfinyl aryl-C₁₋₁₀alkyl, heteroaryl-C₁₋₁₀alkyl, aryl-C₁₋₁₀heteroalkyl, heteroaryl-C₁₋₁₀heteroalkyl, a phosphorus group, a silicon group and a boron group, wherein (Rₐ)₂, (R_{b})₂, (R_{c})₂, or (R_{d})₂ are optionally combined to form a 5 to 8-membered carbocyclic or heterocyclic ring; further wherein the aliphatic or aromatic portions of the two substituents are optionally substituted with from 1 to 4 substituents selected from the group consisting of halogen, cyano, nitro, C₁₋₄alkyl, C₂₋₆alkenyl, C₂₋₆alkynyl, aryl, C₁₋₆alkoxy, C₂₋₆alkenyloxy, C₂₋₆alkynyloxy, aryloxy, C₂₋₆alkoxycarbonyl, C₁₋₆ alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus groups; and
amine is one or more amine groups reacted together or sequentially with the compound of formula (110), the amine being selected from the group consisting of a primary, secondary and tertiary amines.

An additional embodiment of the present invention is a process for making (S)-2-(2,3-bis(dicyclohexylamino)cycloallylamino)-3-phenylpropan-1-ol hydrochloride salt (5.HCl). This process comprises:

(a) contacting a tetrachlorocyclopropene with dicyclohexylamine for a period of time and under conditions suitable for the tetrachlorocyclopropene and the dicyclohexylamine to react and form a reaction mixture; and

(b) contacting the reaction mixture formed in step (a) with phenylalaninol under conditions suitable to form a (S)-2-(2,3-bis(dicyclohexylamino)cycloallylamino)-3-phenylpropan-1-ol hydrochloride salt (5.HCl).

Another embodiment of the present invention is a process for making (S)-2-(2,3-bis(dicyclohexylamino)cycloallylamino)-3-phenylpropan-1-ol. This process comprises:

(a) contacting a tetrachlorocyclopropene with a dicyclohexylamine for a period of time and under conditions suitable for the tetrachlorocyclopropene and the dicyclohexylamine to react and form a reaction mixture;

(b) contacting the reaction mixture formed in step (a) with phenylalaninol under conditions suitable to form (S)-2-(2,3-bis(dicyclohexylamino)cycloallylamino)-3-phenylpropan-1-ol hydrochloride salt (5.HCl);

(c) optionally processing the (S)-2-(2,3-bis(dicyclohexylamino)cycloallylamino)-3-phenylpropan-1-ol hydrochloride salt (5.HCl) formed in step (b) by carrying out one or more of filtering, washing, and drying of the (S)-2-(2,3-bis(dicyclohexylamino)cycloallylamino)-3-phenylpropan-1-ol hydrochloride salt (5.HCl); and

(d) washing the (S)-2-(2,3-bis(dicyclohexylamino)cycloallylamino)-3-phenylpropan-1-ol hydrochloride salt (5.HCl) formed in step (c) with an aqueous organic base.

Yet another embodiment of the present invention is a process for carrying out an organic synthetic reaction. This process comprises:

(a) contacting an organic nucleophile with an organic electrophile in the presence of a cyclopropenimine Brønsted base catalyst under conditions suitable to form a new organic species through the reaction of the organic nucleophile with the organic electrophile, the cyclopropenimine Brønsted base having the structure:

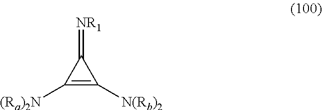

wherein
R₁ is selected from the group consisting of aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀cycloalkenyl, C₂₋₁₀alkynyl, halogen, aryloxy, heteroaryloxy, C₂₋₁₀alkoxycarbonyl, C₁₋₁₀alkylthio, C₂₋₁₀alkenylthio, C₂₋₁₀alkynylthio, C₁₋₁₀alkylsulfonyl, C₁₋₁₀alkylsulfinyl aryl-C₁₋₁₀alkyl, heteroaryl-C₁₋₁₀alkyl, aryl-C₁₋₁₀heteroalkyl, heteroaryl-C₁₋₁₀heteroalkyl; wherein the aliphatic or aromatic portions of R₁ are optionally substituted with from 1 to 4 substituents selected from the group consisting of halogen, cyano, nitro, C₁₋₄-alkyl, C₂₋₆alkenyl, C₂₋₆alkynyl, aryl, C₁₋₆alkoxy, C₂₋₆alkenyloxy, C₂₋₆alkynyloxy, aryloxy, C₂₋₆alkoxycarbonyl, C₁₋₆ alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus groups; and
each Rₐ and R_{b} are the same or different and are independently selected from sterically demanding substituents; and

crystalline forms, hydrates, or salts thereof.

An additional embodiment of the present invention is a process for catalyzing a proton transfer reaction enantioselectively for the production of an optically enriched organic product. This reaction comprises:

(a) contacting an organic nucleophile with an organic electrophile in the presence of a cyclopropenimine Brønsted base catalyst under conditions suitable to form a new organic species through the reaction of the organic nucleophile with the organic electrophile, the cyclopropenimine Brønsted base having the structure:

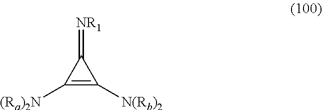

wherein
R₁ is selected from the group consisting of aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀cycloalkenyl, C₂₋₁₀alkynyl, halogen, aryloxy, heteroaryloxy, C₂₋₁₀alkoxycarbonyl, C₁₋₁₀alkylthio, C₂₋₁₀alkenylthio, C₂₋₁₀alkynylthio, C₁₋₁₀alkylsulfonyl, C₁₋₁₀alkylsulfinyl aryl-C₁₋₁₀alkyl, heteroaryl-C₁₋₁₀alkyl, aryl-C₁₋₁₀heteroalkyl, heteroaryl-C₁₋₁₀heteroalkyl; wherein the aliphatic or aromatic portions of R₁ are optionally substituted with from 1 to 4 substituents selected from the group consisting of halogen, cyano, nitro, C₁₋₄-alkyl, C₂₋₆alkenyl, C₂₋₆alkynyl, aryl, C₁₋₆alkoxy, C₂₋₆alkenyloxy, C₂₋₆alkynyloxy, aryloxy, C₂₋₆alkoxycarbonyl, C₁₋₆ alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus groups; and
Rₐ and R_{b} are independently selected from sterically demanding substituents; and

crystalline forms, hydrates, or salts thereof.

Yet another embodiment of the present invention is a process for catalyzing a proton transfer reaction enantioselectively for the production of an optically enriched organic product. This reaction comprises: contacting an organic nucleophile with an organic electrophile in the presence of a 2,3-bis(dialkylamino)-cyclopropenimine under conditions suitable to form a new organic species through the reaction of the organic nucleophile with the organic electrophile.

### BRIEF DESCRIPTION OF THE DRAWINGS

FIG. 1 shows cyclopropenimines as strong Brønsted bases.

FIG. 2 shows the basicity of cyclopropenimine 1 and several common strong organic bases. Bold numbers are pK_{BH+} values in acetonitrile.

FIG. 3 shows the X-ray structure of protonated cyclopropenimine 5.HCl co-crystallized with a molecule of H₂O.

FIG. 4 shows the stereochemical rationale for cyclopropenimine 5 catalyzed enantioselective conjugate addition of compound 2 to methyl acrylate.

FIG. 5 shows the decomposition pathway of cyclopropenimine 5.

FIG. 6 shows the synthesis scheme of cyclopropenimine compound

FIG. 7 shows the synthesis scheme of (S)-2-(2,3-bis(dicyclohexylamino)cycloallylamino)-3-phenylpropan-1-ol (cyclopropenimine compound 5).

FIG. 8 shows the synthesis scheme of (S)—N¹,N¹,N²,N²-tetracyclohexyl-3-(1-phenylethylimino)cycloprop-1-ene-1,2-diamine (cyclopropenimine compound 7).

FIG. 9 shows the synthesis scheme of (S)-2-(2,3-bis(diisopropylamino)cycloallylamino)-3-phenylpropan-1-ol (cyclopropenimine compound 8).

FIG. 10 shows the rearrangement of cyclopropenimine compound 5.

FIG. 11 shows the general procedure for glycinate-Michael Additions.

FIG. 12 shows the synthesis scheme of compound 42.

FIG. 13 shows the synthesis scheme of compound 45.

FIG. 14 shows the synthesis scheme of compound 48.

FIG. 15 shows the synthesis scheme of compound 50.

### DETAILED DESCRIPTION OF THE INVENTION

One embodiment of the present invention is a cyclopropenimine Brønsted base catalyst. This catalyst has the structure:

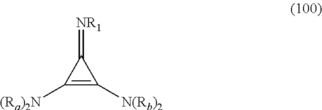

wherein

R₁ is independently selected from the group consisting of aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀cycloalkenyl, C₂₋₁₀alkynyl, halogen, aryloxy, heteroaryloxy, C₂₋₁₀alkoxycarbonyl, C₁₋₁₀alkylthio, C₂₋₁₀alkenylthio, C₂₋₁₀alkynylthio, C₁₋₁₀alkylsulfonyl, C₁₋₁₀alkylsulfinyl aryl-C₁₋₁₀alkyl, heteroaryl-C₁₋₁₀alkyl, aryl-C₁₋₁₀heteroalkyl, heteroaryl-C₁₋₁₀heteroalkyl; wherein the aliphatic or aromatic portions of R₁ are optionally substituted with from 1 to 4 substituents selected from the group consisting of halogen, cyano, nitro, C₁₋₄-alkyl, C₂₋₆alkenyl, C₂₋₆alkynyl, aryl, C₁₋₆alkoxy, C₂₋₆alkenyloxy, C₂₋₆alkynyloxy, aryloxy, C₂₋₆alkoxycarbonyl, C₁₋₆ alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus groups; and

each Rₐ and R_{b} are the same or different and are independently selected from sterically demanding substituents; and

crystalline forms, hydrates, or salts thereof.

As used herein, the term “Brønsted base” has its art recognized meaning, i.e., a compound that can accept a hydrogen ion (H⁺).

In the present invention, “sterically demanding substituents” has its art recognized meaning, namely sterically large and bulky groups. Such substituents include, for example, groups having 3-50 C atoms, such as, phenyl, diphenylmethyl, triphenylmethyl, (di)-isopropyl, t-butyl, neo-pentyl, (di)-cyclohexyl, cycloheptyl or aryl, which, in turn, can be substituted themselves. Suitable substituents in this respect comprise halogen, nitro, and also alkyl or alkoxyl, as well as cycloalkyl or aryl, e.g. phenyl, toluoylmethyl, ditoluoylmethyl and tritoluoylmethyl. Phenyl, benzyl, triphenylmethyl, t-butyl, neo-pentyl, cyclohexyl, fluorenyl, anthracenyl, phenanthrenyl, bromophenyl, chlorophenyl, toluoyl and nitrophenyl may also be used. Also preferred are cyclic or branched, especially alpha, alpha-di-branched and, e.g., alpha-branched, alkyl groups. Optionally, the two Rₐ groups, or the two R_{b} groups may be combined to form a 5 to 8 membered carbocyclic or heterocyclic ring.

In one aspect of this embodiment, R₁ is substituted with a hydroxy.

In another aspect of this embodiment, the sterically demanding substituent are linear, branched, or cyclic alkyl substituents.

Preferably, the cyclopropenimine Brønsted base catalyst has a structure selected from the group consisting of:

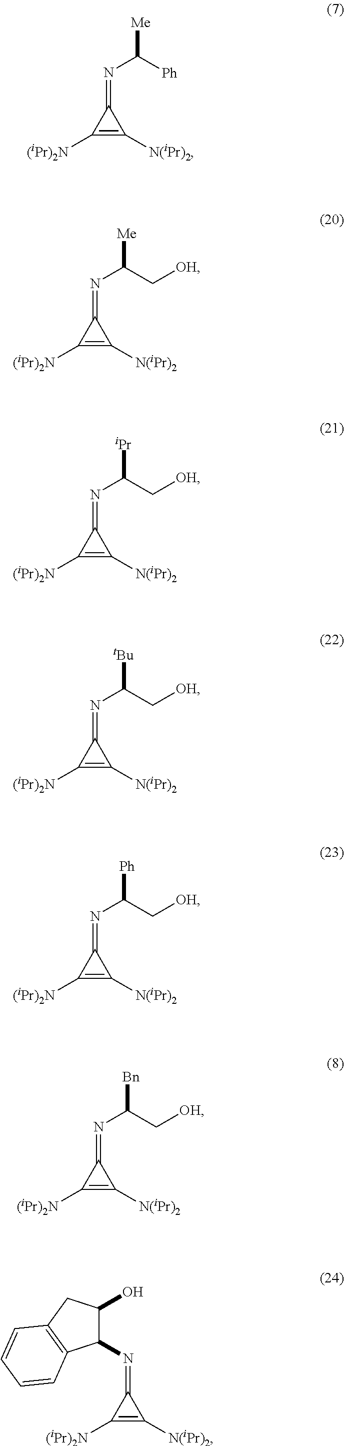

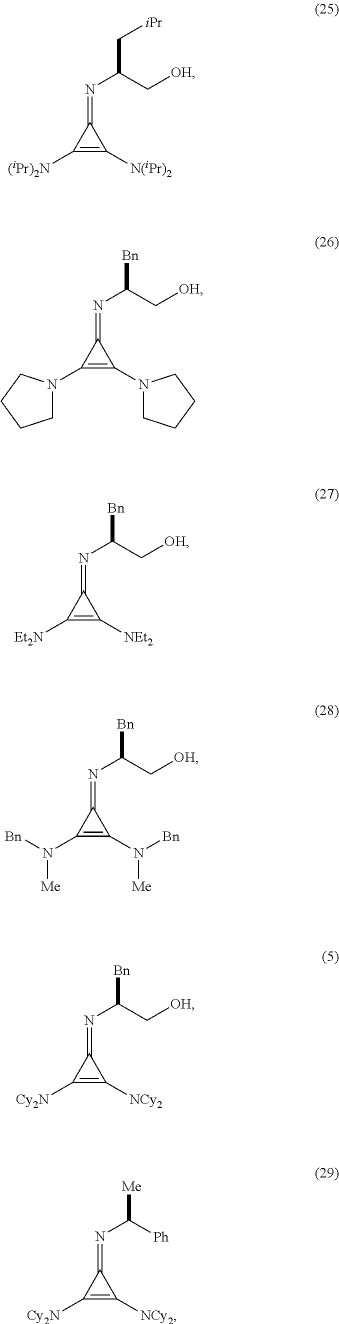

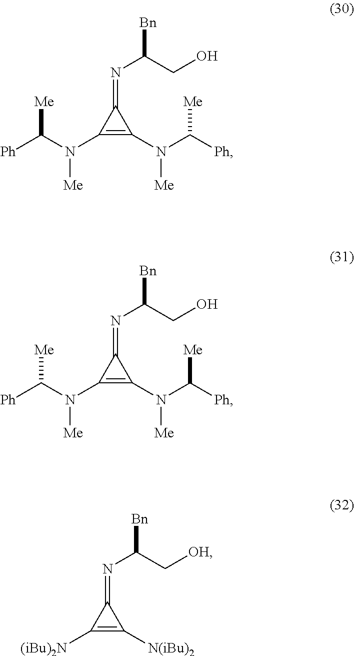

and crystalline forms, hydrates, and salts thereof.

In another preferred embodiment, the cyclopropenimine Brønsted base catalyst is (S)-2-(2,3-bis(dicyclohexylamino)cycloallylamino)-3-phenylpropan-1-ol hydrochloride salt (5.HCl):

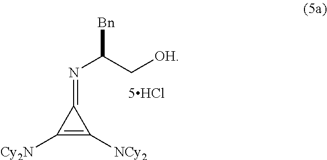

Another embodiment of the present invention is a cyclopropenimine scaffold for use as a Brønsted base catalyst, which forms a cyclopropenium ion upon protonation of an imino nitrogen. This scaffold has the structure:

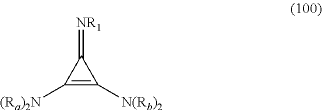

wherein

R₁ is a hydroxyl containing substituent; and

each Rₐ and R_{b} are the same or different and are independently selected from sterically demanding substituents; and

crystalline forms, hydrates, or salts thereof.

Yet another embodiment of the present invention is a process for making a cyclopropenimine suitable for use as a Brønsted base catalyst. This process comprises contacting a compound of formula (110) with an amine under conditions suitable to form a cyclopropenimine Brønsted base catalyst of formula (100):

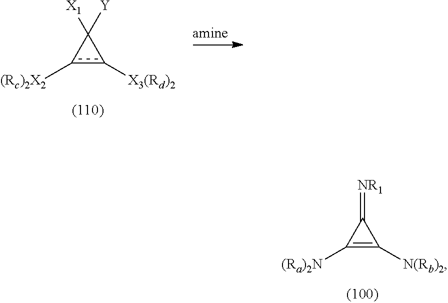

wherein
each X₁₋₃ and Y are the same or different and are independently selected from the group consisting of Cl, N, and no atom;
each Rₐ, R_{b}, R_{c} and R_{d} are the same or different and are independently selected from the group consisting of no atom, amino, aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀cycloalkenyl, C₂₋₁₀alkynyl, halogen, aryloxy, heteroaryloxy, C₂₋₁₀alkoxycarbonyl, C₁₋₁₀alkylthio, C₂₋₁₀alkenylthio, C₂₋₁₀alkynylthio, C₁₋₁₀alkylsulfonyl, C₁₋₁₀alkylsulfinyl aryl-C₁₋₁₀alkyl, heteroaryl-C₁₋₁₀alkyl, aryl-C₁₋₁₀heteroalkyl, heteroaryl-C₁₋₁₀heteroalkyl, a phosphorus group, a silicon group and a boron group, wherein (Rₐ)₂, (R_{b})₂, (R_{c})₂, or (R_{d})₂ are optionally combined to form a 5 to 8-membered carbocyclic or heterocyclic ring; further wherein the aliphatic or aromatic portions of the two substituents are optionally substituted with from 1 to 4 substituents selected from the group consisting of halogen, cyano, nitro, C₁₋₄alkyl, C₂₋₆alkenyl, C₂₋₆alkynyl, aryl, C₁₋₆alkoxy, C₂₋₆alkenyloxy, C₂₋₆alkynyloxy, aryloxy, C₂₋₆alkoxycarbonyl, C₁₋₆ alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus groups; and
amine is one or more amine groups reacted together or sequentially with the compound of formula (110), the amine being selected from the group consisting of a primary, secondary and tertiary amines.

Conditions suitable to form a cyclopropenimine Brønsted base catalyst according to the process shown above are as exemplified herein and are also known in the art, such as those disclosed by Yoshida, 1973. Such conditions include concentrations of the reactants, the duration of the reaction, the temperature of the reaction, various solvents, and other reagents for washing or otherwise purifying the products.

Preferred cyclopropenimine Brønsted base catalysts are as disclosed above.

In one aspect of this embodiment, the compound of formula (110) is

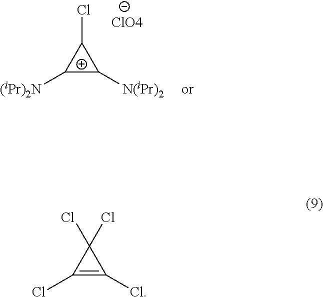

In another aspect of this embodiment, the amine may be any amine capable of participating in the process. For example, the amine may be independently selected from the group consisting of tert-Butylamine (^{t}BuNH₂), dicyclohexylamine (HNCy₂), (S)-2-amino-3-phenylpropan-1-ol, and (S)-1-phenylethanamine.

An additional embodiment of the present invention is a process for making (S)-2-(2,3-bis(dicyclohexylamino)cycloallylamino)-3-phenylpropan-1-ol hydrochloride salt (5.HCl). This process comprises:

(a) contacting a tetrachlorocyclopropene with dicyclohexylamine for a period of time and under conditions suitable for the tetrachlorocyclopropene and the dicyclohexylamine to react and form a reaction mixture; and

(b) contacting the reaction mixture formed in step (a) with phenylalaninol under conditions suitable to form (S)-2-(2,3-bis(dicyclohexylamino)cycloallylamino)-3-phenylpropan-1-ol hydrochloride salt (5.HCl).

Non-limiting exemplary “conditions suitable” for this process are disclosed in the Examples herein and may be further apparent to those skilled in the art in view of the disclosures herein.

Another embodiment of the present invention is a process for making (S)-2-(2,3-bis(dicyclohexylamino)cycloallylamino)-3-phenylpropan-1-ol. This process comprises:

(a) contacting a tetrachlorocyclopropene with a dicyclohexylamine for a period of time and under conditions suitable for the tetrachlorocyclopropene and the dicyclohexylamine to react and form a reaction mixture;

(b) contacting the reaction mixture formed in step (a) with phenylalaninol under conditions suitable to form (S)-2-(2,3-bis(dicyclohexylamino)cycloallylamino)-3-phenylpropan-1-ol hydrochloride salt (5.HCl);

(c) optionally processing the (S)-2-(2,3-bis(dicyclohexylamino)cycloallylamino)-3-phenylpropan-1-ol hydrochloride salt (5.HCl) formed in step (b) by carrying out one or more of filtering, washing, and drying of the (S)-2-(2,3-bis(dicyclohexylamino)cycloallylamino)-3-phenylpropan-1-01 hydrochloride salt (5.HCl); and

(d) washing the (S)-2-(2,3-bis(dicyclohexylamino)cycloallylamino)-3-phenylpropan-1-ol hydrochloride salt (5.HCl) formed in step (c) with an aqueous organic base.

Non-limiting exemplary conditions suitable for this process are disclosed in the Examples herein and may be further apparent to those skilled in the art in view of the disclosures herein.

Yet another embodiment of the present invention is a process for carrying out an organic synthetic reaction. This process comprises:

(a) contacting an organic nucleophile with an organic electrophile in the presence of a cyclopropenimine Brønsted base catalyst under conditions suitable to form a new organic species through the reaction of the organic nucleophile with the organic electrophile, the cyclopropenimine Brønsted base having the structure:

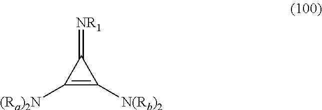

wherein
R₁ is selected from the group consisting of aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀cycloalkenyl, C₂₋₁₀alkynyl, halogen, aryloxy, heteroaryloxy, C₂₋₁₀alkoxycarbonyl, C₁₋₁₀alkylthio, C₂₋₁₀alkenylthio, C₂₋₁₀alkynylthio, C₁₋₁₀alkylsulfonyl, C₁₋₁₀alkylsulfinyl aryl-C₁₋₁₀alkyl, heteroaryl-C₁₋₁₀alkyl, aryl-C₁₋₁₀heteroalkyl, heteroaryl-C₁₋₁₀heteroalkyl; wherein the aliphatic or aromatic portions of R₁ are optionally substituted with from 1 to 4 substituents selected from the group consisting of halogen, cyano, nitro, C₁₋₄alkyl, C₂₋₆alkenyl, C₂₋₆alkynyl, aryl, C₁₋₆alkoxy, C₂₋₆alkenyloxy, C₂₋₆alkynyloxy, aryloxy, C₂₋₆alkoxycarbonyl, C₁₋₆ alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus groups; and
each Rₐ and R_{b} are the same or different and are independently selected from sterically demanding substituents; and

crystalline forms, hydrates, or salts thereof.

As used herein, an organic “electrophile” means an organic compound that forms a bond to its reaction partner (the nucleophile) by accepting both bonding electrons from that reaction partner. An organic “nucleophile” means an organic compound that forms a bond to its reaction partner (the electrophile) by donating both bonding electrons. The present invention contemplates use of any organic electrophile and any organic nucleophile so long as their reaction to form a new organic species can be catalyzed by a cyclopropenimine Brønsted base according to the present invention.

Preferred cyclopropenimine Brønsted bases are as disclosed above.

In one aspect of this embodiment, the R₁ of the cyclopropenimine Brønsted base (100) is substituted with a hydroxy.

As used herein “conditions suitable” to form a new organic species means those conditions that can result in the formation of a new covalent bond between the reactants. Non-limiting exemplary conditions suitable for this process are disclosed in the Examples herein and may be further apparent to those skilled in the art in view of the disclosures herein.

The organic synthetic reaction of the present embodiment may be a conjugate addition reaction. As used herein, “conjugated addition reactions” means a reaction in which a nucleophile reacts with a α,β-unsaturated carbonyl compound in the β position. Such a nucleophile may be a carbon nucleophile or a heteratom nucleophiles including nitrogen, sulfur, and phosphorus nucleophiles. Exemplary conjugated addition reactions include those in which carbonyls react with secondary amines to form 1,4-keto-amines, those in which conjugated carbonyls react with hydrogen cyanide to 1,4-keto-nitriles, the Michael reaction, and the Stork enamine reaction involving the conjugate addition of enamines to conjugated carbonyls.

Additional representative organic synthetic reactions according to the present invention also include a Mannich reaction, a Michael reaction, an α-heterofunctionalization of a carbonyl reaction, a Henry (nitroaldol) reaction, an Aza-Henry reaction, a Strecker reaction, a kinetic resolution reaction, and a desymmetrization reaction. Preferably, the reaction is a Michael reaction. Another preferred reaction is a Mannich reaction.

As used herein, “Mannich reaction” means an amino alkylation of an acidic proton placed next to a carbonyl functional group with formaldehyde and ammonia or any primary or secondary amine. Mannich reactions include the reaction between an enolizable carbonyl compound and an azomethine function, which yields a β-amino carbonyl product

As used herein, “Michael reactions” means the 1,4-addition of a carbon nucleophile to an α,β-unsaturated carbonyl compound.

As used herein, “α-heterofunctionalization of a carbonyl reaction” means the transformation of a C—H group adjacent to a carbonyl into a C—X group, wherein X is an electrophile. Such reactions include α-oxygenation (Christoffers et al., 2004; Merino et al., 2004; Plietker, 2005; Janey, 2005), α-aminations (Duthaler, 2003; Greck et al., 2004), α-sulfenylations, and α-halogenations (Ibrain et al., 2004; Oetrech, 2005; Pihko, 2006; Prakash, 2006).

As used herein, a “Henry (nitroaldol) reaction” means a base-catalyzed C—C bond-forming reaction between nitroalkanes and aldehydes or ketones. It is also referred to as the Nitroaldol Reaction.

As used herein, an “aza-Henry reaction” means the addition of nitroalkanes to imines.

As used herein, a “Strecker reaction” means the condensation of aldehydes with ammonia and hydrogen cyanide to form α-amino nitriles followed by hydrolysis of the nitrile group. It is one of the methods for the de novo synthesis of α-amino acids.

As used herein, a “kinetic resolution” means a reaction in which two enantiomers show different reaction rates in a chemical reaction, thereby creating an excess of the less reactive enantiomer.

As used herein, a “desymmetrization reaction” means a process for selection of enantiomers in which the reaction takes place faster at one of the enantiotopic groups or faces of a particular meso or a prochiral compound, thus yielding the two enantiomers of the product in unequal amounts. A “prochiral” compound is one that contains, or is bonded to, two constitutionally identical ligands (atoms or groups), replacement of one of which by a different ligand makes the molecule or atom chiral. A “meso” compound is superimposed on its mirror image and optically inactive although it contains two or more stereocenters.

In one aspect of this embodiment, the Michael reaction is carried out according to the following scheme:

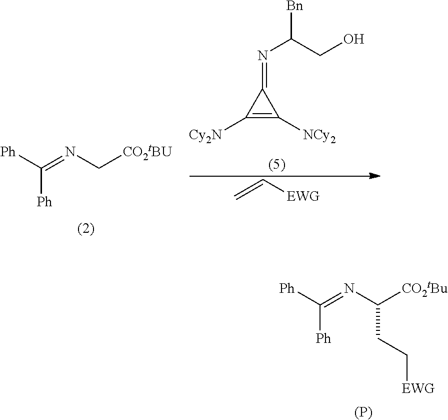

under conditions sufficient to produce a product (P), wherein the EWG is an electronic withdrawing group suitable for participating in the reaction.

As used herein, an “electron withdrawing group” or “EWG” means an atom or group that draws electron density from neighboring atoms towards itself. The mechanism for such EWG include resonance or inductive effects. In the present invention, “EWG” includes, without limitation, the following exemplary compounds:

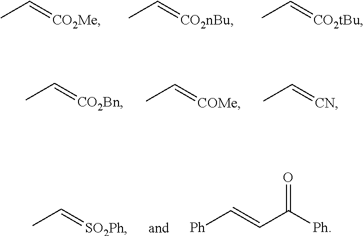

In another aspect of this embodiment, the Mannich reaction is carried out according to the following scheme:

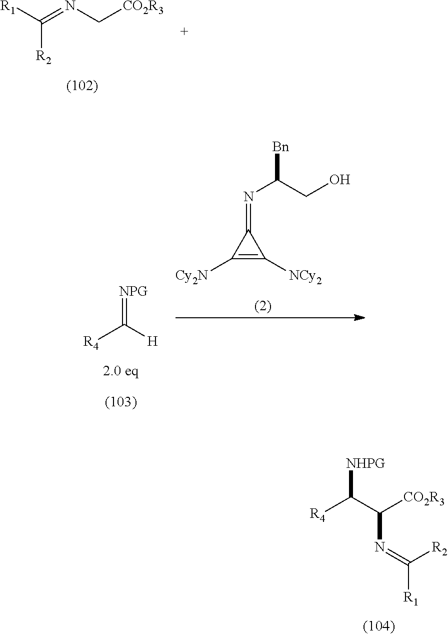

under conditions sufficient to produce a product (104), wherein
R₁, R₂, R₃, R₄ are independently selected from the group consisting of H, C₁₋₆ alkyl, aryl, and heteroaryl; and
PG is a protecting group suitable for participating in the reaction.

As used herein, a “protecting group” or “PG” means an atom or a group of atoms that are used in synthesis to temporarily mask the characteristic chemistry of a functional group. In the present invention, “PG” includes, without limitation, any protecting group known to those skilled in the art and which will not unduly interfere with the Mannich reaction. Exemplary PGs according to the present invention include carbobenzyloxy (Cbz), p-methoxybenzyl carbonyl (Moz or MeOZ), tert-butyloxycarbonyl (BOC), 9-fluorenylmethyloxycarbonyl (FMOC), acetyl (Ac) group, benzoyl (Bz), benzyl (Bn), carbamate, p-methoxybenzyl (PMB), 3,4-dimethoxybenzyl (DMPM), p-methoxyphenyl (PMP), and sulfonamide (such as tosyl (Ts)). Preferably, the PG is BOC.

In one preferred embodiment, R₁ and R₂ are independently selected from the group consisting of H, phenyl, and 4-chlorophenyl. In another preferred embodiment, R₃ is selected from the group consisting of tert-butyl, methyl, and benzyl. In an additional preferred embodiment, R₄ is selected from the group consisting of:

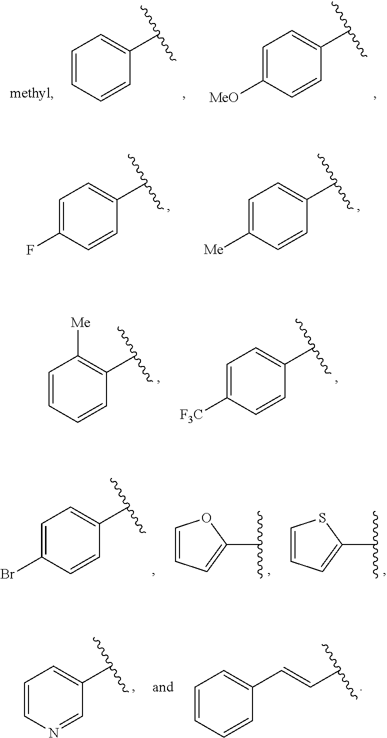

While preferred R₁, R₂, R₃, and R₄ groups are identified above, any group, particularly those disclosed herein, may be used so long as the selected groups do not unduly interfere with the Mannich reaction.

More preferably, the product (104) is selected from the group consisting of:

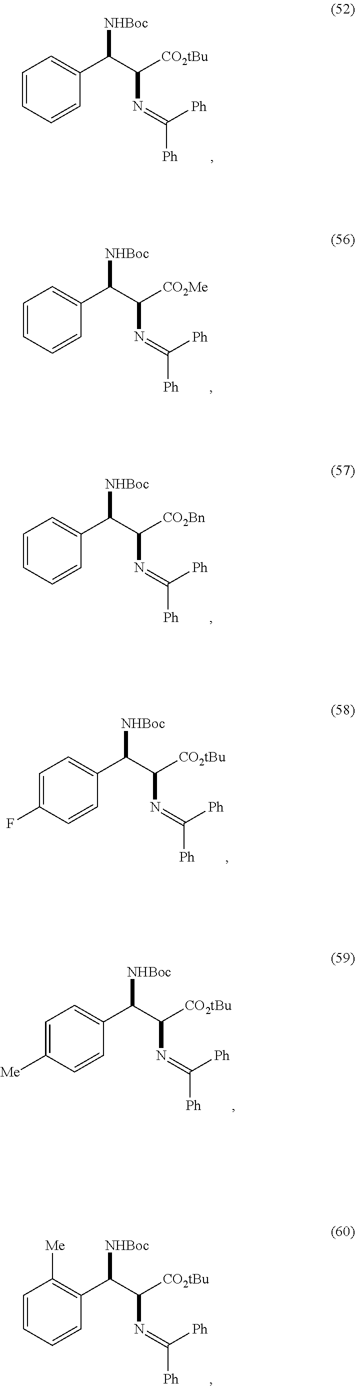 [SMILES: CC(C)(C)OC(=O)N[C@H](c1ccccc1)[C@H](N=C(c1ccccc1)c1ccccc1)C(=O)OC(C)(C)C ; COC(=O)[C@@H](N=C(c1ccccc1)c1ccccc1)[C@H](NC(=O)OC(C)(C)C)c1ccccc1 ; CC(C)(C)OC(=O)N[C@H](c1ccccc1)[C@H](N=C(c1ccccc1)c1ccccc1)C(=O)OCc1ccccc1 ; CC(C)(C)OC(=O)N[C@H](c1ccc(F)cc1)[C@H](N=C(c1ccccc1)c1ccccc1)C(=O)OC(C)(C)C ; Cc1ccc([C@@H](NC(=O)OC(C)(C)C)[C@H](N=C(c2ccccc2)c2ccccc2)C(=O)OC(C)(C)C)cc1 ; Cc1ccccc1[C@@H](NC(=O)OC(C)(C)C)[C@H](N=C(c1ccccc1)c1ccccc1)C(=O)OC(C)(C)C]

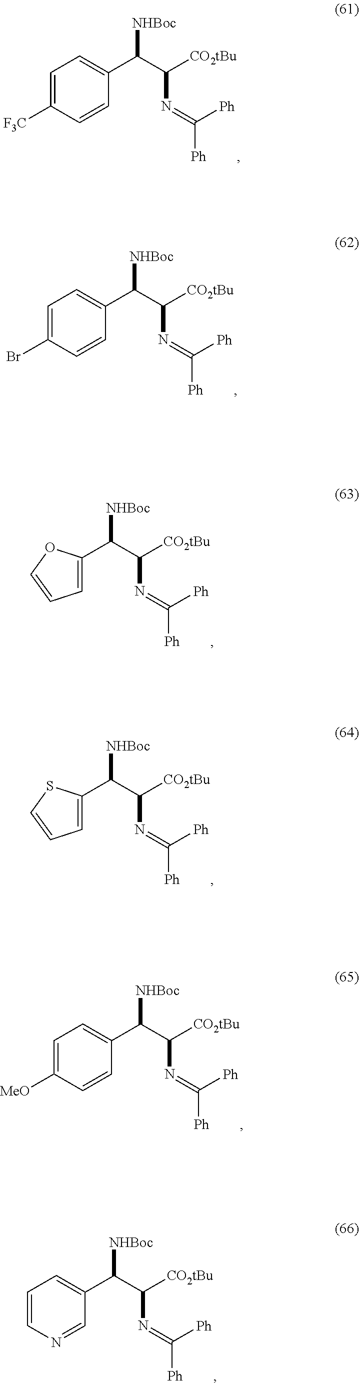 [SMILES: CC(C)(C)OC(=O)N[C@H](c1ccc(C(F)(F)F)cc1)[C@H](N=C(c1ccccc1)c1ccccc1)C(=O)OC(C)(C)C ; CC(C)(C)OC(=O)N[C@H](c1ccc(Br)cc1)[C@H](N=C(c1ccccc1)c1ccccc1)C(=O)OC(C)(C)C ; CC(C)(C)OC(=O)N[C@H](c1ccco1)[C@H](N=C(c1ccccc1)c1ccccc1)C(=O)OC(C)(C)C ; CC(C)(C)OC(=O)N[C@H](c1cccs1)[C@H](N=C(c1ccccc1)c1ccccc1)C(=O)OC(C)(C)C ; COc1ccc([C@@H](NC(=O)OC(C)(C)C)[C@H](N=C(c2ccccc2)c2ccccc2)C(=O)OC(C)(C)C)cc1 ; CC(C)(C)OC(=O)N[C@H](c1cccnc1)[C@H](N=C(c1ccccc1)c1ccccc1)C(=O)OC(C)(C)C]

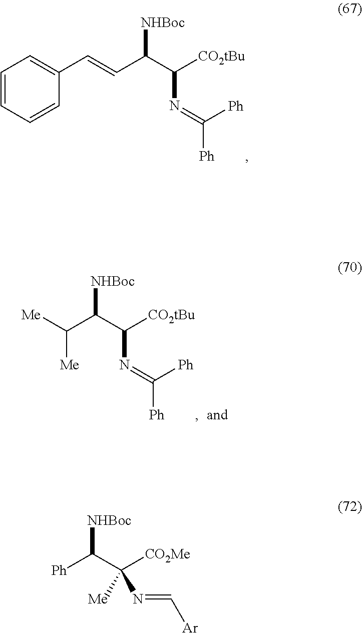

Preferred cyclopropenimine Brønsted base catalysts are as disclosed above. In another preferred embodiment, the cyclopropenimine Brønsted base catalyst has the structure:

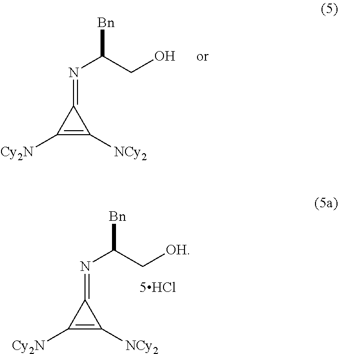

An additional embodiment of the present invention is a process for catalyzing a proton transfer reaction enantioselectively for the production of an optically enriched organic product. This reaction comprises:

(a) contacting an organic nucleophile with an organic electrophile in the presence of a cyclopropenimine Brønsted base catalyst under conditions suitable to form a new organic species through the reaction of the organic nucleophile with the organic electrophile, the cyclopropenimine Brønsted base having the structure:

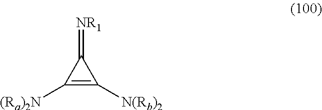

wherein
R₁ is selected from the group consisting of aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀cycloalkenyl, C₂₋₁₀alkynyl, halogen, aryloxy, heteroaryloxy, C₂₋₁₀alkoxycarbonyl, C₁₋₁₀alkylthio, C₂₋₁₀alkenylthio, C₂₋₁₀alkynylthio, C₁₋₁₀alkylsulfonyl, C₁₋₁₀alkylsulfinyl aryl-C₁₋₁₀alkyl, heteroaryl-C₁₋₁₀alkyl, aryl-C₁₋₁₀heteroalkyl, heteroaryl-C₁₋₁₀heteroalkyl; wherein the aliphatic or aromatic portions of R₁ are optionally substituted with from 1 to 4 substituents selected from the group consisting of halogen, cyano, nitro, C₁₋₄-alkyl, C₂₋₆alkenyl, C₂₋₆alkynyl, aryl, C₁₋₆alkoxy, C₂₋₆alkenyloxy, C₂₋₆alkynyloxy, aryloxy, C₂₋₆alkoxycarbonyl, C₁₋₆ alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus groups; and
Rₐ and R_{b} are independently selected from sterically demanding substituents; and

crystalline forms, hydrates, or salts thereof.

As used herein, a “proton transfer reaction” means a chemical reaction in which a proton is intermolecularly or intramolecularly transferred from one site of attachment to another.

As used herein, “enantioselectively” means that one enantiomer of a chiral product is preferentially produced as a result of the reaction. The present reaction optically enriches the organic product. Thus, the reaction may be an optically pure product or a racemic mixture of products, which is enriched for one of the products. In the case where an optically pure or substantially optically pure product is desired, the desired enantiomer may be further purified by any of the methods disclosed in more detail below.

Preferred cyclopropenimine Brønsted base catalysts are as disclosed above.

Yet another embodiment of the present invention is a process for catalyzing a proton transfer reaction enantioselectively for the production of an optically enriched organic product. This reaction comprises: contacting an organic nucleophile with an organic electrophile in the presence of a 2,3-bis(dialkylamino)-cyclopropenimine under conditions suitable to form a new organic species through the reaction of the organic nucleophile with the organic electrophile.

Preferably, the 2,3-bis(dialkylamino)-cyclopropenimine has a structure selected from the group consisting of:

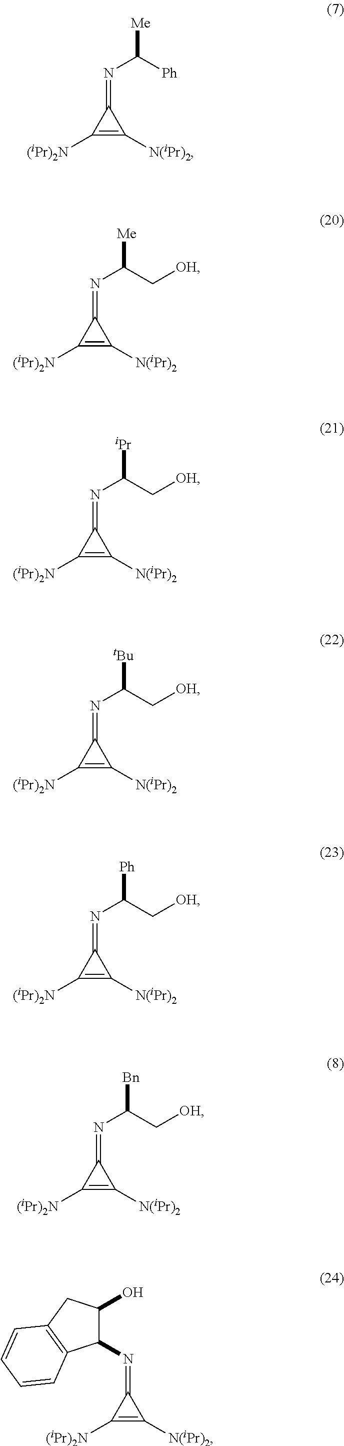

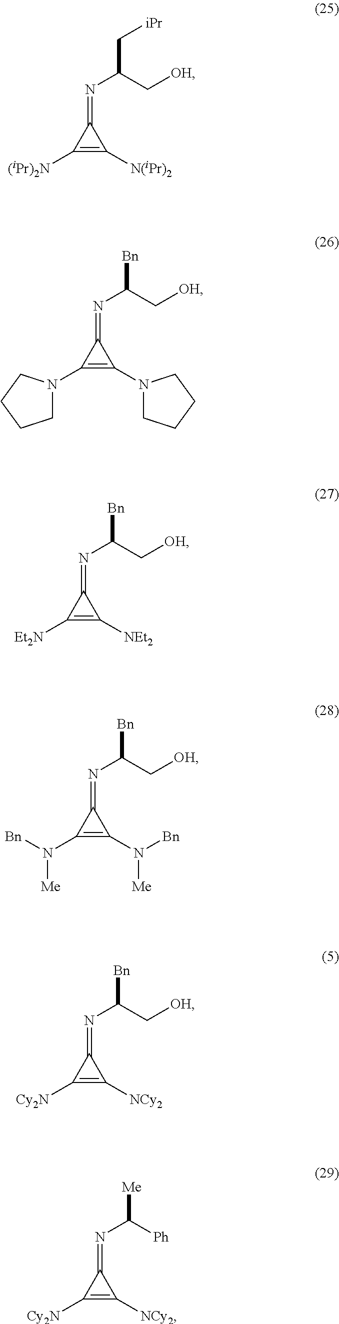

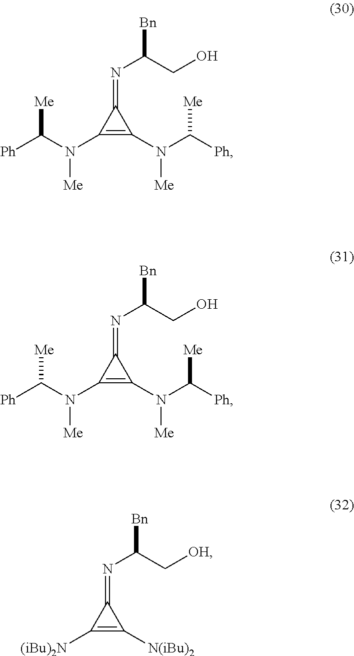

and crystalline forms, hydrates, or salts thereof.

More preferably, the 2,3-bis(dialkylamino)-cyclopropenimine has the structure:

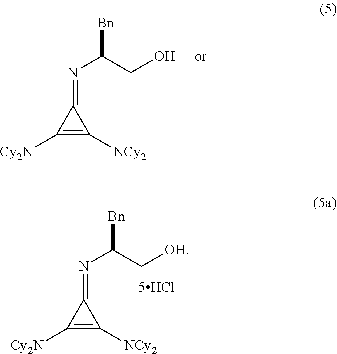

In the foregoing embodiments, the following definitions apply.

As used herein, the term “acyl” has its art-recognized meaning and refers to a group represented by the general formula hydrocarbylC(O)—, preferably alkylC(O)—.

As used herein, the term “acylamino” has its art-recognized meaning and refers to an amino group substituted with an acyl group and may be represented, for example, by the formula hydrocarbylC(O)NH—.

The term “aldehyde” refers to s an organic compound containing a functional group with the structure R—CHO.

The term “alkoxy” refers to an alkyl group, preferably a lower alkyl group, having an oxygen attached thereto. Representative alkoxy groups include methoxy, ethoxy, propoxy, isopropoxy, tert-butoxy and the like. Other alkoxy groups within the scope of the present invention include, for example, the following:

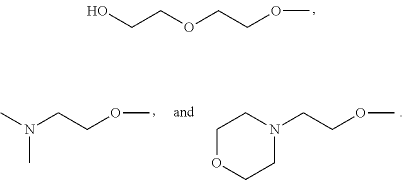

The term “alkoxyalkyl” refers to an alkyl group substituted with an alkoxy group and may be represented by the general formula alkyl-O-alkyl.

The term “alkoxycarbonyl” refers to a carbonyl group substituted with an alkoxy group.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, substitution of alkenyl groups by one or more alkyl, carbocyclyl, aryl, heterocyclyl, or heteroaryl groups is contemplated.

The term “alkenyloxy” refers to an alkenyl group having an oxygen attached thereto.

The term “alkenylthio”, as used herein, refers to a thiol group substituted with an alkenyl group and may be represented by the general formula alkenylS—.

The term “alkyl” refers to the radical of saturated aliphatic groups, including straight-chain alkyl groups, branched-chain alkyl groups, cycloalkyl (alicyclic) groups, alkyl-substituted cycloalkyl groups, and cycloalkyl-substituted alkyl groups. In certain embodiments, a straight chain or branched chain alkyl has 10 or fewer carbon atoms in its backbone (e.g., C₁-C₁₀ for straight chains, C₃-C₁₀ for branched chains). Likewise, certain cycloalkyls have from 3-8 carbon atoms in their ring structure, including 5, 6 or 7 carbons in the ring structure.

Moreover, unless otherwise indicated, the term “alkyl” (or “lower alkyl”) as used throughout the specification, examples, and claims is intended to include both “unsubstituted alkyls” and “substituted alkyls”, the latter of which refers to alkyl moieties having substituents replacing a hydrogen on one or more carbons of the hydrocarbon backbone. Such substituents can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aromatic, or heteroaromatic moiety. It will be understood by those skilled in the art that the moieties substituted on the hydrocarbon chain can themselves be substituted, if appropriate. For instance, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “C_{x-y},” when used in conjunction with a chemical moiety, such as, alkyl, alkenyl, or alkoxy is meant to include groups that contain from x to y carbons in the chain. For example, the term “C_{x-y}alkyl” refers to substituted or unsubstituted saturated hydrocarbon groups, including straight-chain alkyl and branched-chain alkyl groups that contain from x to y carbons in the chain, including haloalkyl groups such as trifluoromethyl and 2,2,2-tirfluoroethyl, etc. The terms “C_{2-y}alkenyl” and “C_{2-y}alkynyl” refer to substituted or unsubstituted unsaturated aliphatic groups analogous in length and possible substitution to the alkyls described above, but that contain at least one double or triple bond respectively.

The term “alkylamino”, as used herein, refers to an amino group substituted with at least one alkyl group.

The term “alkylsulfinyl” means a sulfinyl group substituted with an alkyl group.

The term “alkylsulfonyl” means a sulfonyl group substituted with an alkyl group.

The term “alkylthio”, as used herein, refers to a thiol group substituted with an alkyl group and may be represented by the general formula alkylS—.

The term “alkynyl”, as used herein, refers to an aliphatic group containing at least one triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more triple bonds. Moreover, such substituents include all those contemplated for alkyl groups, as discussed above, except where stability is prohibitive. For example, substitution of alkynyl groups by one or more alkyl, carbocyclyl, aryl, heterocyclyl, or heteroaryl groups is contemplated.

The term “alkynyloxy” means an alkynyl group having an oxygen attached thereto.

The term “alkynylthio”, as used herein, refers to a thiol group substituted with an alkynyl group and may be represented by the general formula alknnylS—.

The term “amide” or “amido”, as used herein, refers to a group

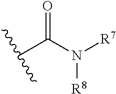

wherein R⁷ and R⁸ each independently represent a hydrogen or hydrocarbyl group, or R⁷ and R⁸ taken together with the N atom to which they are attached complete a heterocycle having from 4 to 8 atoms in the ring structure.

The terms “amine” and “amino” are art-recognized and refer to both unsubstituted and substituted amines and salts thereof, e.g., a moiety that can be represented by

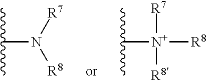

wherein R⁷, R⁸, and R^{8′} each independently represent a hydrogen or a hydrocarbyl group, or R⁷ and R⁸ taken together with the N atom to which they are attached complete a heterocycle having from 4 to 8 atoms in the ring structure. The term “primary” amine means only one of R⁷ and R⁸ or one of R⁷, R⁸, and R^{8′} is a hydrocarbyl group. Secondary amines have two hydrocarbyl groups bound to N. In tertiary amines, all three groups, R⁷, R⁸, and R^{8′}, are replaced by hydrocarbyl groups.

The term “amino acid,” as used herein, refers a functional group containing both amine and carboxyl groups.

The term “aminoalkyl”, as used herein, refers to an alkyl group substituted with an amino group.

The term “aralkyl”, as used herein, refers to an alkyl group substituted with an aryl group.

The term “aryl” as used herein includes substituted or unsubstituted single-ring aromatic groups in which each atom of the ring is carbon. Preferably the ring is a 3- to 8-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

As used herein, “aryloxy” means which an aryl group singularly bonded to oxygen.

The term “aryl-alkyl” means an alkyl group substituted with aryl.

The term “aryl-heteroalkyl” means an heteroalkyl group substituted with aryl.

As used herein, “azomethine” is a compound with a functional group that contains a carbon-nitrogen double bond with the nitrogen atom connected to an aryl or alkyl group, not hydrogen.

The term “carbamate” is art-recognized and refers to a group

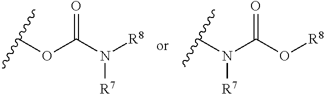

wherein R⁷ and R⁸ independently represent hydrogen or a hydrocarbyl group.

The terms “carbocycle”, “carbocyclyl”, and “carbocyclic”, as used herein, refer to a non-aromatic saturated or unsaturated ring in which each atom of the ring is carbon. Preferably a carbocycle ring contains from 3 to 8 atoms, including 5 to 7 atoms, such as for example, 6 atoms.

The term “carbonyl” means a functional group composed of a carbon atom double-bonded to an oxygen atom: C═O. Carbonyls include without limitation, aldehydes, ketones, carboxylic acids, esters, and amides.

As used herein “α,β-unsaturated carbonyl compound” means a carbonyl compound with the general structure Cβ=Cα-(C═O)—. In these compounds the carbonyl group is conjugated with an alkene.

The terms “carboxy” and “carboxyl”, as used herein, refer to a group represented by the formula —CO₂H.

The term “carboxylate” refers to the conjugate base of a carboxyl group, represented by the formula —COO⁻.

The term “cycloalkyl” means a univalent groups derived from cycloalkanes by removal of a hydrogen atom from a ring carbon atom.

The term “cycloalkenyl” means a univalent groups derived from cycloalkenes by removal of a hydrogen atom from a ring carbon atom.

The term “crystalline” forms, as used herein, refers to the crystal structure of a compound. A compound may exist in one or more crystalline forms, which may have different structural, physical, pharmacological, or chemical characteristics. Different crystalline forms may be obtained using variations in nucleation, growth kinetics, agglomeration, and breakage. Nucleation results when the phase-transition energy barrier is overcome, thereby allowing a particle to form from a supersaturated solution. Crystal growth is the enlargement of crystal particles caused by deposition of the chemical compound on an existing surface of the crystal. The relative rate of nucleation and growth determine the size distribution of the crystals that are formed. The thermodynamic driving force for both nucleation and growth is supersaturation, which is defined as the deviation from thermodynamic equilibrium. Agglomeration is the formation of larger particles through two or more particles (e.g., crystals) sticking together and forming a larger crystalline structure.

The term “enol”, as used herein, are alkenes with a hydroxyl group affixed to one of the carbon atoms composing the double bond. “Enolate anions” are deprotonated anions of enols. An “enolizable” carbonyl means a carbonyl that has an α-proton and be able to undergo deprotonation to form the enolate anion.

The term “ester”, as used herein, refers to a group —C(O)OR⁷ wherein R⁷ represents a hydrocarbyl group.

The term “ether”, as used herein, refers to a hydrocarbyl group linked through an oxygen to another hydrocarbyl group. Accordingly, an ether substituent of a hydrocarbyl group may be hydrocarbyl-O—. Ethers may be either symmetrical or unsymmetrical. Examples of ethers include, but are not limited to, heterocycle-O-heterocycle and aryl-O-heterocycle. Ethers include “alkoxyalkyl” groups, which may be represented by the general formula alkyl-O-alkyl.

The terms “halo” and “halogen” are used interchangeably herein and mean halogen and include chloro, fluoro, bromo, and iodo.

The term “heteroaryl” includes substituted or unsubstituted aromatic single ring structures, preferably 3- to 8-membered rings, more preferably 5- to 7-membered rings, even more preferably 5- to 6-membered rings, whose ring structures include at least one heteroatom, preferably one to four heteroatoms, more preferably one or two heteroatoms. The term “heteroaryl” also includes polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

As used herein, “heteroaryloxy” means which a heteroaryl group singularly bonded to oxygen.

“Heteroaryl-heteroalkyl” means a heteroalkyl group substituted with a heteroaryl group.

The term “heteroatom” as used herein means an atom of any element other than carbon or hydrogen. Preferred heteroatoms are nitrogen, oxygen, and sulfur.

The term “heteroalkyl” means an alkyl in which at least one carbon of a hydrocarbon backbone is substituted with a heteroatom. Heteroalkyls include alkoxyalkyls, such as C₁₋₈ alkoxyalkyl.

The term “heteroaromatic” means at least one carbon atoms in the aromatic group is substituted with a heteroatom.

The terms “heterocyclyl”, “heterocycle”, “heterocyclic”, and the like refer to substituted or unsubstituted non-aromatic ring structures, preferably 3- to 8-membered rings, whose ring structures include at least one heteroatom, preferably one to four heteroatoms, more preferably one or two heteroatoms. The terms “heterocyclyl,” “heterocyclic,” and the like also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “hydrates”, as used herein, refers to a solid or a semi-solid form of a chemical compound containing water in a molecular complex. The water is generally in a stoichiometric amount with respect to the chemical compound.

The term “hydrocarbyl”, as used herein, refers to a group that is bonded through a carbon atom that does not have a ═O or ═S substituent, and typically has at least one carbon-hydrogen bond and a primarily carbon backbone, but may optionally include heteroatoms. Thus, groups like methyl, ethoxyethyl, 2-pyridyl, and trifluoromethyl are considered to be hydrocarbyl for the purposes of this application, but substituents such as acetyl (which has a ═O substituent on the linking carbon) and ethoxy (which is linked through oxygen, not carbon) are not. Hydrocarbyl groups include, but are not limited to aryl, heteroaryl, carbocycle, heterocycle, alkyl, alkenyl, alkynyl, and combinations thereof.

The term “hydroxyalkyl”, as used herein, refers to an alkyl group substituted with a hydroxy group.

The term “hydroxyl” or “hydroxy,” as used herein, refers to the group —OH.

The term “imine” means a chemical compound containing an imino group.

The term “imino” group means a functional group containing a carbon-nitrogen double bond.

The term “imino nitrogen”, as used herein, means a nitrogen in the imino group.

The term “ketone” means an organic compound with the structure RC(═O)R′, wherein neither R and R′ can be hydrogen atoms.

The term “lower” when used in conjunction with a chemical moiety, such as, acyl, alkyl, alkenyl, alkynyl, or alkoxy is meant to include groups where there are ten or fewer non-hydrogen atoms in the substituent, preferably eight or fewer, such as for example, from about 2 to 8 carbon atoms, including less than 6 carbon atoms. A “lower alkyl”, for example, refers to an alkyl group that contains ten or fewer carbon atoms, preferably eight or fewer. In certain embodiments, acyl, alkyl, alkenyl, alkynyl, or alkoxy substituents defined herein are respectively lower acyl, lower alkyl, lower alkenyl, lower alkynyl, or lower alkoxy, whether they appear alone or in combination with other substituents, such as in the recitations hydroxyalkyl and aralkyl (in which case, for example, the atoms within the aryl group are not counted when counting the carbon atoms in the alkyl substituent).

As used herein, a “nitroalkane” mean an alkane with a NO₂ group attached to one end.

As used herein, a “nitrile” means any organic compound that has a —C≡N functional group.

The terms “polycyclyl”, “polycycle”, and “polycyclic” refer to two or more rings (e.g., cycloalkyls, cycloalkenyls, cycloalkynyls, aryls, heteroaryls, and/or heterocyclyls) in which two or more atoms are common to two adjoining rings, e.g., the rings are “fused rings”. Each of the rings of the polycycle can be substituted or unsubstituted. In certain embodiments, each ring of the polycycle contains from 3 to 10 atoms in the ring, preferably from 3 to 8, such as for example, 5 to 7.

The term “oxo” refers to the group ═O.

As used herein, the term “organic” refers to any carbon-based compound. In addition to carbon, organic compounds may contain calcium, chlorine, fluorine, hydrogen, potassium, nitrogen, oxygen, sulfur and other elements. An organic compound as used herein also includes nitrated organic compounds and halogenated (e.g., chlorinated) organic compounds.

The term “substituted” refers to moieties having substituents replacing a hydrogen on one or more carbons of the backbone. It will be understood that “substitution” or “substituted with” includes the implicit proviso that such substitution is in accordance with the permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. It will be understood by those skilled in the art that the moieties substituted on the hydrocarbon chain can themselves be substituted, if appropriate.

As used herein, the term “substituent,” means H, cyano, oxo, nitro, acyl, acylamino, halogen, hydroxy, amino acid, amine, amide, carbamate, ester, ether, carboxylic acid, thio, thioalkyl, thioester, thioether, C₁₋₈ alkyl, C₁₋₈alkoxy, C₁₋₈alkenyl, C₁₋₈aralkyl, 3- to 8-membered carbocyclic, 3- to 8-membered heterocyclic, 3- to 8-membered aryl, or 3- to 8-membered heteroaryl, sulfate, sulfonamide, sulfoxide, sulfonate, sulfone, alkylsulfonyl, and arylsulfonyl.

Unless specifically stated as “unsubstituted,” references to chemical moieties herein are understood to include substituted variants. For example, reference to an “aryl” group or moiety implicitly includes both substituted and unsubstituted variants.

The term “sulfate” is art-recognized and refers to the group —OSO₃H, or a pharmaceutically acceptable salt thereof.

The term “sulfonamide” is art-recognized and refers to the group represented by the general formulae

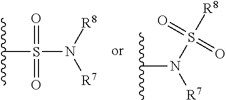

wherein R⁷ and R⁸ independently represents hydrogen or hydrocarbyl.

The term “sulfinyl” is art-recognized and refers to the group —S(O)—R⁷, wherein R⁷ represents a hydrocarbyl.

The term “sulfonate” is art-recognized and refers to the group SO₃H, or a pharmaceutically acceptable salt thereof.

The term “sulfonyl” is refers to the group —S(O)₂—R⁷, wherein R⁷ represents a hydrocarbyl.

The term “thio” or “thiol”, as used herein, refers to the —SH group.

The term “thioalkyl”, as used herein, refers to an alkyl group substituted with a thiol group.

The term “thioester”, as used herein, refers to a group —C(O)SR⁷ or —SC(O)R⁷ wherein R⁷ represents a hydrocarbyl.

The term “thioether”, as used herein, is equivalent to an ether, wherein the oxygen is replaced with a sulfur.

The term “thiono” refers to a substitution on a carbon atom, more specifically to a doubly bonded sulfur.

It is understood that the disclosure of a compound herein encompasses all stereoisomers of that compound. As used herein, the term “stereoisomer” refers to a compound made up of the same atoms bonded by the same bonds but having different three-dimensional structures which are not interchangeable. The three-dimensional structures are called configurations. Stereoisomers include enantiomers, optical isomers, and diastereomers.

The terms “racemate” or “racemic mixture” refer to a mixture of equal parts of enantiomers. The term “chiral center” refers to a carbon atom to which four different groups are attached. The term “enantiomeric enrichment” as used herein refers to the increase in the amount of one enantiomer as compared to the other.

It is appreciated that compounds of the present invention having a chiral center may exist in and be isolated in optically active and racemic forms. Some compounds may exhibit polymorphism. It is to be understood that the present invention encompasses any racemic, optically-active, diastereomeric, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

Examples of methods to obtain optically active materials are known in the art, and include at least the following:
i) physical separation of crystals—a technique whereby macroscopic crystals of the individual enantiomers are manually separated. This technique can be used if crystals of the separate enantiomers exist, i.e., the material is a conglomerate, and the crystals are visually distinct;
ii) simultaneous crystallization—a technique whereby the individual enantiomers are separately crystallized from a solution of the racemate, possible only if the latter is a conglomerate in the solid state;
iii) enzymatic resolutions—a technique whereby partial or complete separation of a racemate by virtue of differing rates of reaction for the enantiomers with an enzyme;
iv) enzymatic asymmetric synthesis—a synthetic technique whereby at least one step of the synthesis uses an enzymatic reaction to obtain an enantiomerically pure or enriched synthetic precursor of the desired enantiomer;
v) chemical asymmetric synthesis—a synthetic technique whereby the desired enantiomer is synthesized from an achiral precursor under conditions that produce asymmetry (i.e., chirality) in the product, which may be achieved using chiral catalysts as disclosed in more detail herein or chiral auxiliaries;
vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;
vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;
viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;
ix) enantiospecific synthesis from non-racemic precursors—a synthetic technique whereby the desired enantiomer is obtained from non-chiral starting materials and where the stereochemical integrity is not or is only minimally compromised over the course of the synthesis;
x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;
xi) chiral gas chromatography—a technique whereby the racemate is volatilized and enantiomers are separated by virtue of their differing interactions in the gaseous mobile phase with a column containing a fixed non-racemic chiral adsorbent phase;
xii) extraction with chiral solvents—a technique whereby the enantiomers are separated by virtue of preferential dissolution of one enantiomer into a particular chiral solvent;
xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

The stereoisomers may also be separated by usual techniques known to those skilled in the art including fractional crystallization of the bases or their salts or chromatographic techniques such as LC or flash chromatography. The (+) enantiomer can be separated from the (−) enantiomer using techniques and procedures well known in the art, such as that described by J. Jacques, et al., antiomers, Racemates, and Resolutions”, John Wiley and Sons, Inc., 1981. For example, chiral chromatography with a suitable organic solvent, such as ethanol/acetonitrile and Chiralpak AD packing, 20 micron can also be utilized to effect separation of the enantiomers.

The following examples are provided to further illustrate the compounds, compositions, and processes of the present invention. These examples are illustrative only and are not intended to limit the scope of the invention in any way.

### EXAMPLES

### Example 1

### 2,3-bis(dialkylamino)cyclopropenimines Are Potent Brønsted Bases

Although the principle behind the strong basicity of cyclopropenimines is well appreciated, to the best of inventors' knowledge no actual measurement of this basicity has been reported. Although Maksic and coworkers have calculated that 2,3-bisaminocyclopropenimines should be superbases, with proton affinities that far exceed the corresponding guanidines (256 kcal mol⁻¹ vs. 235 kcal mol⁻¹) (Maksic et al., 1999; Gattin et al., 2005), this prediction had not been experimentally verified previously. Furthermore, although cyclopropenimines have been recently used as a nitrogen(I)-based ligands, the use of cyclopropenimines as reagents or catalysts is also unknown (Bruns et al., 2010).

The inventors have measured the acidity of the conjugate acid (pK_{BH+}) of cyclopropenimine (compound 1),

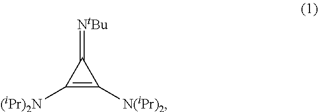

in acetonitrile (26.9) and found it to be comparable to the bicyclic guanidine TBD (26.03) and the phosphazene base P1-^{t}Bu (26.98), both of which are considered to be exceptionally strong “superbases” (Kaljurand et al., 2005). Notably, cyclopropenimine (compound 1) is three orders of magnitude more basic than a comparable guanidine, BTMG (23.56). These findings confirm for the first time that 2,3-bis(dialkylamino)cyclopropenimines are indeed potent Brønsted bases.

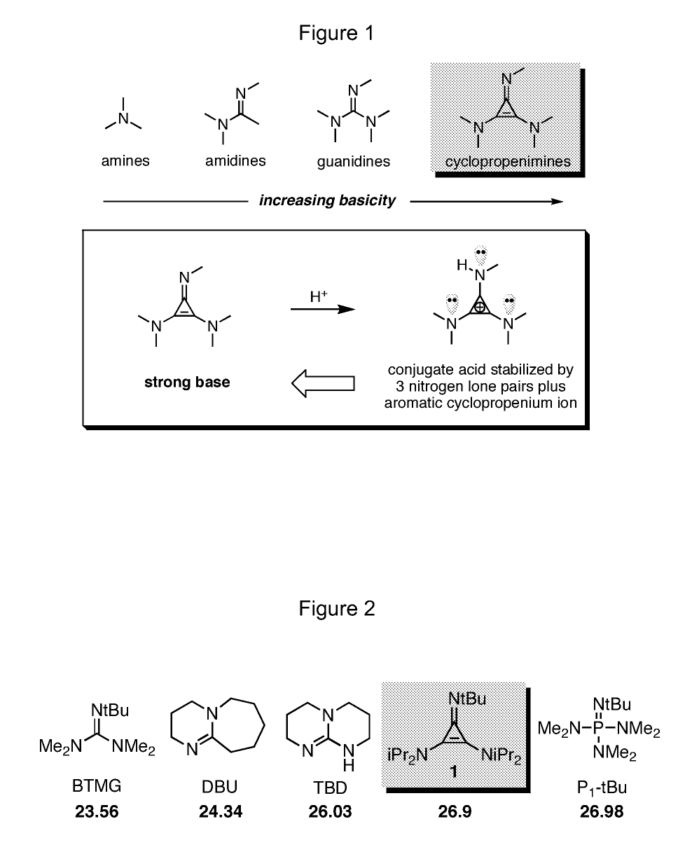

The signature feature of the cyclopropenimine scaffold (Paquette et al., 1967; Eicher et al., 1987; Eicher et al., 1980; Krebs et al., 1984; Weiss et al., 1980; Pilli et al., 1983; Komatsu et al., 2003; Krebs et al., 1965; Yoshida, 1973; D'yakonov, 1967) is the presence of a latent cyclopropenium ion, which is revealed upon protonation of the imino nitrogen (FIG. 1). As the smallest ring system that satisfies Hückel's rules, the 2π-electron cyclopropenium ion (Breslow et al., 1957; Breslow et al., 1958) provides substantial aromatic resonance stability to the conjugate acid of the cyclopropenimine. In comparison to the analogous guanidines, this additional stabilization renders 2,3-bis(dialkylamino)cyclopropenimines highly basic.

### Example 2

### Cyclopropenimines for Brønsted Base-Catalyzed Transformations

The inventors recognized that the strong basicity of cyclopropenimines might offer advantages in terms of reactivity and reaction scope for Brønsted base-catalyzed transformations.

### Michael Reaction

The inventors initially selected the Michael reaction of glycine imine 2 with methyl acrylate 3 as a forum for comparison to reported chiral guanidine catalysts (Hashimoto et al., 2007; O'Donnell et al., 2004).

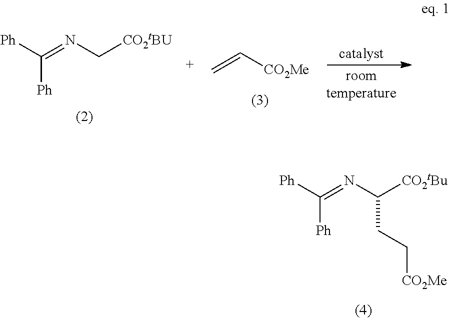

In this regard, cyclopropenimine 5 was identified

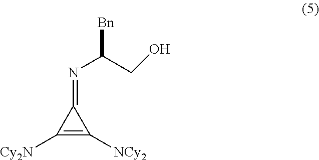

as a highly effective catalyst, which at 10 mol % loading effects the production of adduct 4 in essentially quantitative yield and 91% enantiomer excess (“ee”) in only 5 minutes under neat conditions. When the reaction was performed in ethyl acetate, a product was obtained in quantitative yield and with 98% ee in 1 hour. In comparison to the high performance of this cyclopropenimine catalyst, reported chiral guanidine catalysts have been far less effective.

For example, 20 mol % of guanidine 6

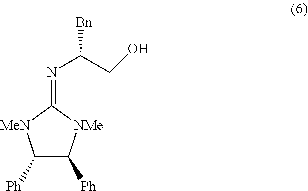 [SMILES: CN1C(=N[C@@H](CO)Cc2ccccc2)N(C)[C@@H](c2ccccc2)[C@@H]1c1ccccc1]

catalyzes the production of 4 in high yield and good ee only after 3 days reaction time at high concentration (neat) (Ishikawa et al., 2001; Ryoda et al., 2008; Isobe et al., 2000a; Isobe et all, 2000b; Isobe et al., 2000c; Zhang et al., 2010; Isobe et al., 1998; Isobe et al., 2001; Kumamoto et al., 2005; Kitani et al., 2005; Saito et al., 2008). Notably, these guanidine catalyzed reactions have not been viable in solution. This comparison clearly illustrates the potential of cyclopropenimines to serve as a powerful new platform for chiral Brønsted base catalysis.

A selection of the optimization studies were conducted to arrive at the conditions shown in equation 1. These selections are shown in Table 1 below.

TABLE 1
Optimization of enantioselective
cyclopropenimine catalyzed Michael addition^{a}

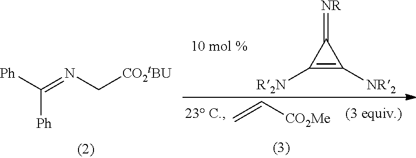

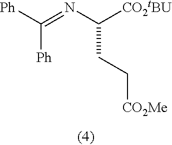

| | catalyst | | | time | conv. | ee |
|---|---|---|---|---|---|---|
| entry | (mol %) | solvent (M) | | (h) | (%) | (%) |
| 1 | 5 (10) | THF | (0.20) | 24 | 86 | 89 |
| 2 | 5 (10) | Et₂O | (0.20) | 2 | >95 | 98 |
| 3 | 5 (10) | dioxane | (0.20) | 8 | >95 | 98 |
| 4 | 5 (10) | MeCN | (0.20) | 1 | >95 | 28 |
| 5 | 5 (10) | EtCO₂Me | (0.20) | 10 | >95 | 95 |
| 6 | 5 (10) | EtOAc | (0.20) | 2 | >95 | 98 |
| 7 | 5 (10) | EtOAc | (0.35) | 1 | >95 | 98 |
| 8 | 5 (5) | EtOAc | (0.35) | 6 | >95 | 98 |
| 9 | 5 (2.5) | EtOAc | (0.35) | 18 | >95 | 97 |
| 10 | 5 (1) | EtOAc | (0.35) | 24 | 78 | 97 |
| 11 | 7 (10) | EtOAc | (0.35) | 24 | 11 | 0 |
| 12 | 8 (10) | EtOAc | (0.35) | 24 | 40 | 87 |

| | |
|---|---|
| 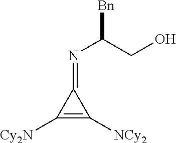 | (5) |
| 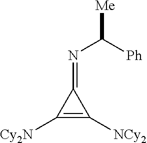 | (7) |
| 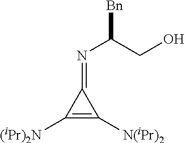 | (8) |

^{a}Conversion determined by ¹H NMR versus Bn₂O standard.

In terms of solvent, ethers including THF, Et₂O, and dioxane were viable media for this process using catalyst 5 (entries 1-3 of Table 1 above) albeit with significant variation in reaction time. The reaction was fast in acetonitrile but enantioselectivity was greatly compromised (entry 4 of Table 1 above), which may be a reflection of the propensity for this solvent to engage in hydrogen bonding and thus to disrupt transition state organization. On the other hand, ester solvents such as methyl propionate (entry 5 of Table 1 above) and, optimally, ethyl acetate (entry 6 of Table 1 above) proved to be most convenient. Reactions can be significantly accelerated by increasing concentration, with a concentration of 0.35 M resulting in the optimized reaction shown in equation 1 (entry 7 of Table 1 above). Reduction of catalyst loading down to 2.5 mol % could be achieved without loss of conversion or enantioselectivity (entries 8-9 of Table 1 above). Even the use of only 1 mol % catalyst was possible although, in this case, the conversion after 24 hours was reduced to 78% (entry 10 of Table 1 above).

In terms of catalyst structure, the presence of a hydroxyl group was crucial for both reactivity and enantioselectivity, with catalysts such as 7 producing low conversions of product with 0% ee (entry 11 of Table 1 above). Sterically demanding dialkylamino substituents at the 2 and 3 positions were also found to be important for optimal performance. For example, the diisopropylamine derived catalyst 8 was markedly less efficient and selective than catalyst 5 under the same conditions (entry 12 of Table 1 above).

A screen of Michael acceptors revealed that various acrylate esters are also viable substrates for catalyst 5. Table 2 below shows the substrate scope of Michael acceptors.

TABLE 2
Substrate scope of Michael acceptors.^{a}

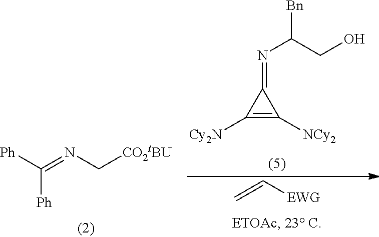

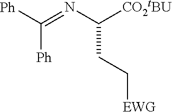

| | | time | | |
|---|---|---|---|---|
| entry | electrophile | (h) | yield (%) | ee (%) |
| 1 | 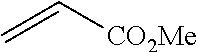 | 1 | 99 | 98 |
| 2 | 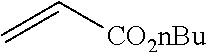 | 1.5 | 99 | 98 |
| 3 | 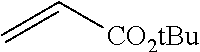 | 12 | 98 | 99 |
| 4 | 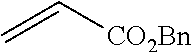 | 1 | 97 | 98 |
| 5 | 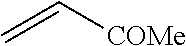 | 0.25 | 97 | 95 |
| 6 | 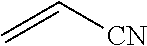 | 30 | 97 | 77 |
| 7b | 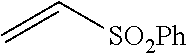 | 24 | 89 | 41 |
| 8 | 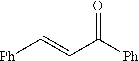 [SMILES: O=C(/C=C/c1ccccc1)c1ccccc1] | 1 | 97 | 95 (6:1 dr) |

^{a}Yield based on isolated and purified product.
^{b}Yield determined by ¹H NMR versus Bn₂O standard.

Thus, in addition to methyl acrylate (entry 1 of Table 2 above), n-butyl, t-butyl, and benzyl acrylates also participated in nearly quantitative yield and with high enantioselectivity (entries 2-4 of Table 2 above). Notably, t-butyl acrylate reacted significantly slower than either methyl or n-butyl acrylate, which is consistent with the hypothesis that interaction of the catalyst with the ester carbonyl via hydrogen bonding plays an important role in catalysis of this reaction. Methyl vinyl ketone was quite reactive, proceeding to full conversion in only 15 minutes (entry 5 of Table 2 above). On the other hand, both acrylonitrile (entry 6 of Table 2 above) and phenyl vinyl sulfone (entry 7 of Table 2 above) reacted dramatically slower and with greatly diminished enantioselectivity. It is believed that differences in hydrogen bonding geometry between these substrates and the carboxylate substrates may account for these disparities. Lastly, a chalcone substrate reacted with high efficiency to produce the Michael addition adduct in high yield and 95% ee as a 6:1 mixture of diastereomers (entry 8 of Table 2 above).

### Mannich Reaction

Cyclopropenimine 5 was also used in the following Mannich Reaction.

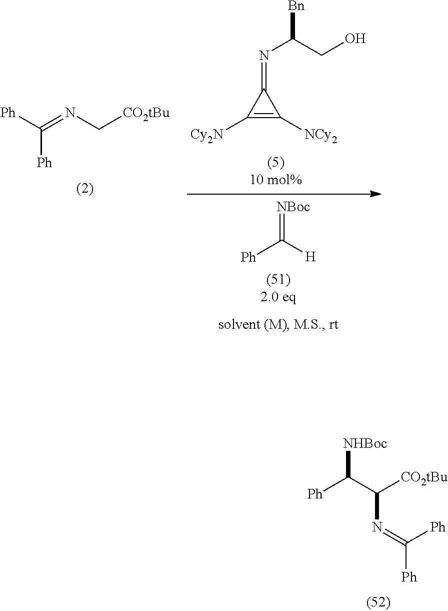

A selection of the optimization studies were conducted, as shown in Table 3 below. All reactions were carried out at room temperature (rt). In some of the reactions, activated molecular sieves (M.S.) was used, as indicated in Table 3.

TABLE 3

| Entry | Solvent (M) | Time (h) | dr (syn/anti) | ee (%) |
|---|---|---|---|---|
| 1 | EtOAc (0.35) | 12 | 98:2 | 83 |
| 2 | Et₂O (0.35) | 8 | 98:2 | 85 |
| 3 | PhH (0.35) | 20 | 99:1 | 89 |
| 4 | m-xylene (0.35) | 20 | 99:1 | 91 |
| 5 | PhMe (0.35) | 16 | 99:1 | 91 |
| 6 | PhMe (0.45) | 6 | 99:1 | 90 |
| 7 | PhMe (0.25) | 24 | 99:1 | 92 |
| 8 | PhMe (0.15) | 36 | 99:1 | 94 |
| 9 | PhMe (0.08) | 48 | 98:2 | 93 |
| 10^{a} | PhMe (0.25) | 20 | 98:2 | 95 |
| 11^{a,b} | PhMe (0.25) | 26 | 98:2 | 95 |
| 12^{a} | PhMe (0.20) | 28 | 98:2 | 94 |

^{a}ground molecular sieves;
^{b}1.5 eq of imine

A screen of various reactants in the Mannich Reaction was performed using catalyst 5. Using the following reaction, products 52 and 56-65 listed in Table 4 below was made, and products 66-67 may be made.

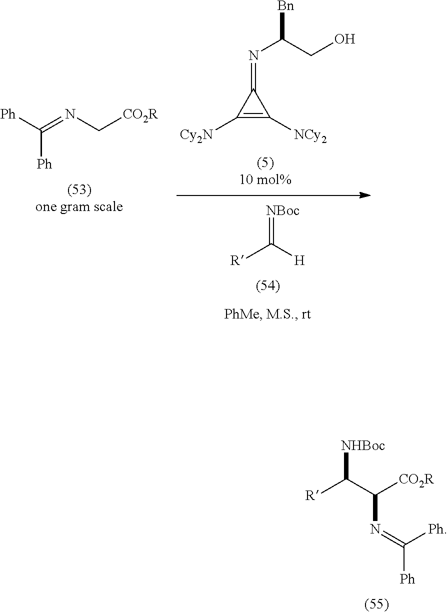

TABLE 4
Mannich reaction products, reaction conditions, and yield

| | |
|---|---|
| 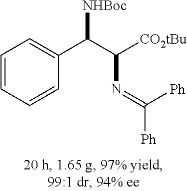 [SMILES: CC(C)(C)OC(=O)N[C@H](c1ccccc1)[C@H](N=C(c1ccccc1)c1ccccc1)C(=O)OC(C)(C)C] | (52) |
| 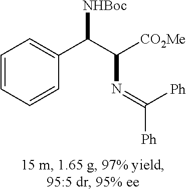 [SMILES: COC(=O)[C@@H](N=C(c1ccccc1)c1ccccc1)[C@H](NC(=O)OC(C)(C)C)c1ccccc1] | (56) |
| 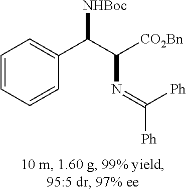 [SMILES: CC(C)(C)OC(=O)N[C@H](c1ccccc1)[C@H](N=C(c1ccccc1)c1ccccc1)C(=O)OCc1ccccc1] | (57) |
| 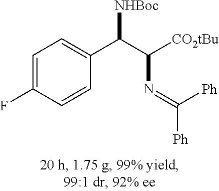 [SMILES: CC(C)(C)OC(=O)N[C@H](c1ccc(F)cc1)[C@H](N=C(c1ccccc1)c1ccccc1)C(=O)OC(C)(C)C] | (58) |
| 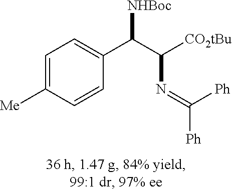 [SMILES: Cc1ccc([C@@H](NC(=O)OC(C)(C)C)[C@H](N=C(c2ccccc2)c2ccccc2)C(=O)OC(C)(C)C)cc1] | (59) |
| 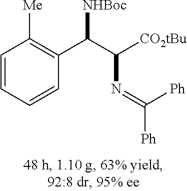 [SMILES: Cc1ccccc1[C@@H](NC(=O)OC(C)(C)C)[C@H](N=C(c1ccccc1)c1ccccc1)C(=O)OC(C)(C)C] | (60) |
| 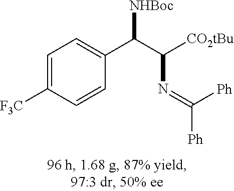 [SMILES: CC(C)(C)OC(=O)N[C@H](c1ccc(C(F)(F)F)cc1)[C@H](N=C(c1ccccc1)c1ccccc1)C(=O)OC(C)(C)C] | (61) |
| 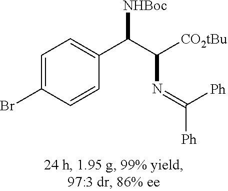 [SMILES: CC(C)(C)OC(=O)N[C@H](c1ccc(Br)cc1)[C@H](N=C(c1ccccc1)c1ccccc1)C(=O)OC(C)(C)C] | (62) |
| 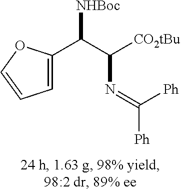 [SMILES: CC(C)(C)OC(=O)N[C@H](c1ccco1)[C@H](N=C(c1ccccc1)c1ccccc1)C(=O)OC(C)(C)C] | (63) |
| 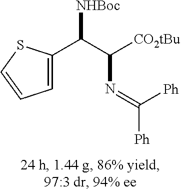 [SMILES: CC(C)(C)OC(=O)N[C@H](c1cccs1)[C@H](N=C(c1ccccc1)c1ccccc1)C(=O)OC(C)(C)C] | (64) |
| 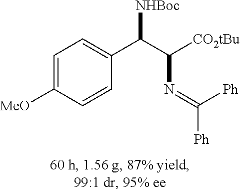 [SMILES: COc1ccc([C@@H](NC(=O)OC(C)(C)C)[C@H](N=C(c2ccccc2)c2ccccc2)C(=O)OC(C)(C)C)cc1] | (65) |
| 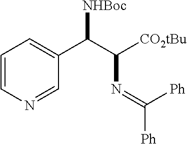 [SMILES: CC(C)(C)OC(=O)N[C@H](c1cccnc1)[C@H](N=C(c1ccccc1)c1ccccc1)C(=O)OC(C)(C)C] | (66) |
| 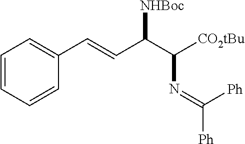 [SMILES: CC(C)(C)OC(=O)N[C@H](/C=C/c1ccccc1)[C@H](N=C(c1ccccc1)c1ccccc1)C(=O)OC(C)(C)C] | (67) |

The following reaction was also carried out:

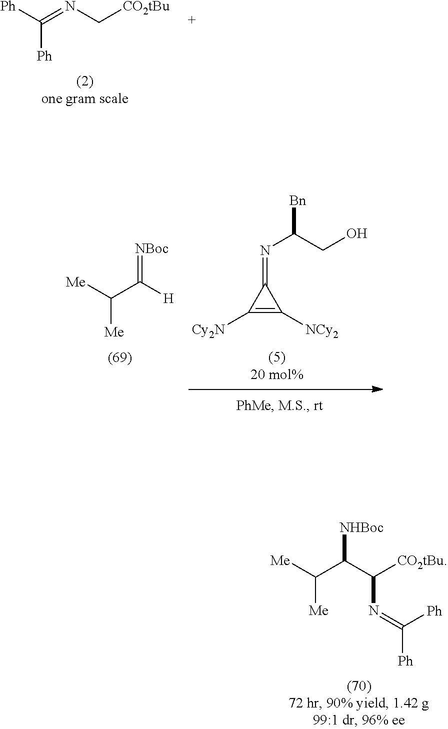

Another reaction using another glycine imine 71 was also performed:

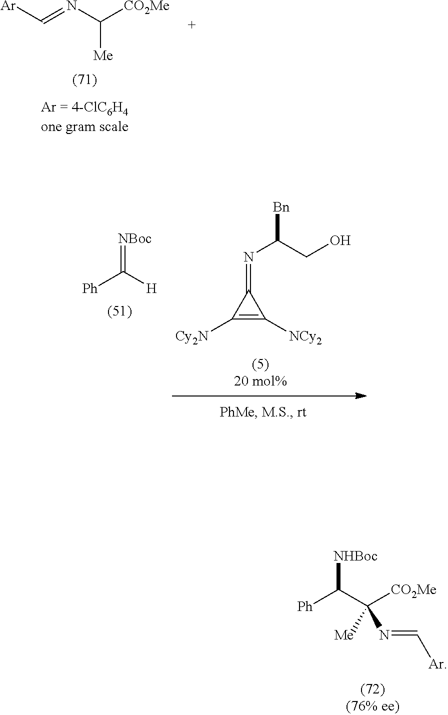

The cyclopropenimine catalyzed Mannich reaction of glycine imines and N-Boc-aldimines is advantageous over previous literature for the following reasons. First, no metal catalyst is used nor required. Second, the protecting group used on the Mannich acceptor is a Boc group, which is easily hydrolyzed, whereas most current methods require tosyl or other sulfonamide protecting groups. For example, Hernandez et al. (2010) used a metal catalyst with a sulfonamide protecting group:

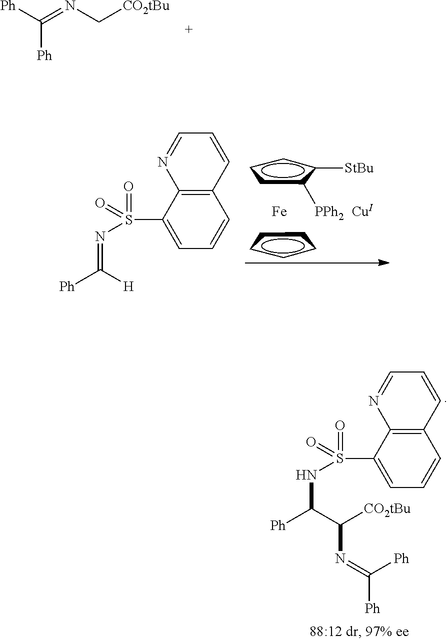

Third, the reactions are carried out on gram-scale and open to air. Fourth, the reactivity of cyclopropenimines is high compared to known organocatalysts, such as guanidine, as disclosed by Kobayashi et al. (2008):

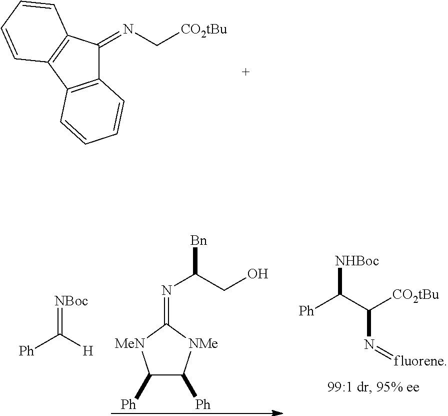

### Example 3

### Features of 2,3-bis(dialkylamino)cyclopropenimines

One of the most attractive features of 2,3-bis(dialkylamino)cyclopropenimines is their extreme ease of synthesis. As a prime example, we have developed a trivial large scale synthesis of catalyst 5 starting from inexpensive and readily available materials (see eq. 2 below) (Yoshida et al., 1971). Thus, tetrachlorocyclopropene (9) (West et al., 1971) was treated with an excess of dicyclohexylamine for 4 hours, followed by the addition of phenylalaninol. From this procedure the salt 5.HCl was isolated in essentially quantitative yield as a crystalline solid.

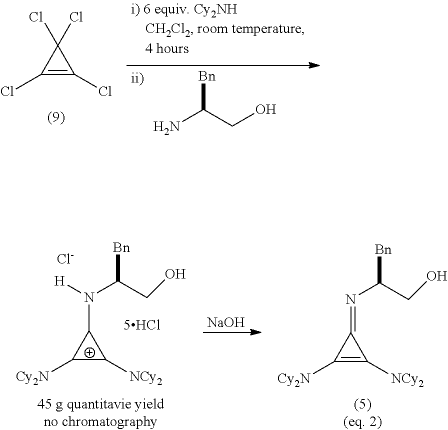

As discussed below, the inventors have found it most convenient to store the catalyst as its HCl salt, and the inventors have prepared as much as 45 g in a single run. The generation of cyclopropenimine 5 requires only a simple wash with aqueous inorganic base and can be used without purification after concentration from solvent.

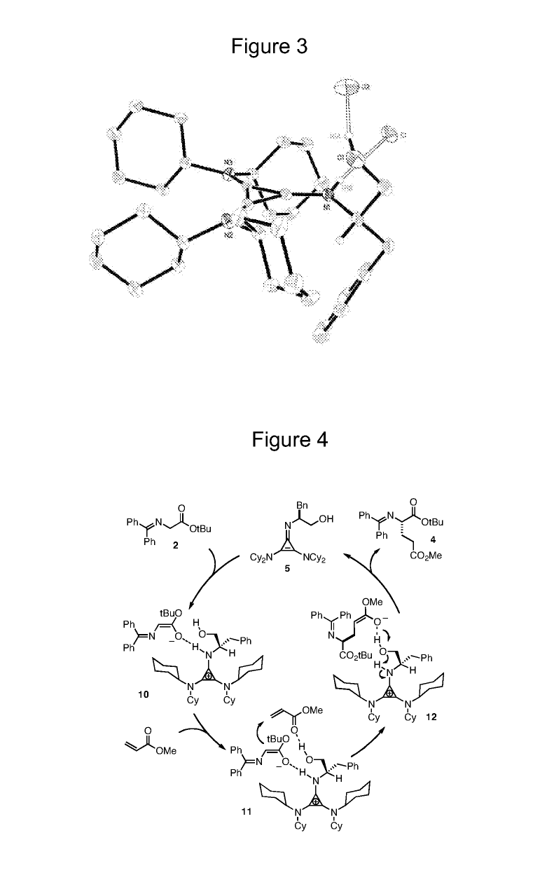

The crystalline nature of 5.HCl allowed an X-ray structure to be obtained (Parkin group, Columbia University), which revealed several key structural features (FIG. 3). First, the steric demand of the dicyclohexylamino groups causes these substituents to torque out of planarity with the cyclopropenium ring. A similar effect has been observed with bis(dialkylamino)cyclopropenyl carbenes (Shoeller et al., 2008). This phenomenon is likely the reason for the diminished basicity of 5 relative to other cyclopropenimines. Second, the chiral appendage appears to be oriented with the proton directed toward a cyclohexyl ring so as to minimize steric conflict. We propose this element of organization is key to the observed high levels of stereocontrol. Finally, it can be seen that the chloride counter ion is hydrogen bonded to the hydrogen-bearing amino group, rather than associated with the cationic cyclopropenium ring, and that a co-crystallized molecule of water is hydrogen bonded to the hydroxyl group. One can readily imagine that such interactions are involved in transition state organization of the conjugate addition of glycine imine 2.

The structure of protonated 5 led the inventors to envision a tentative mechanistic and stereochemical rationale for this transformation (FIG. 4). Thus the inventors believe that the catalyst 5 deprotonates glycine imine 2 to generate the cyclopropenium enolate salt 10. This complex is presumed to involve a hydrogen bond with the N—H of the cyclopropenium, but the precise organization of this salt 10 is at this time unknown. Nevertheless, because the alcohol moiety of the catalyst was found to be required for enantioselectivity, it was speculated that methyl acrylate is directed for conjugate attack via hydrogen bonding with the pendant hydroxyl (cf. 11). Rapid proton transfer from the cyclopropenium ion to the resultant enolate as shown in structure 12 then closes the catalytic cycle.

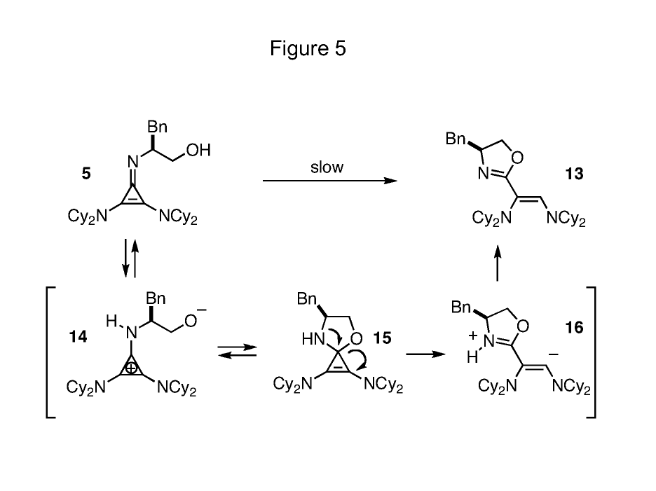

It should be noted that the activity of catalyst 5 was observed to slowly diminish over several days when stored at room temperature. Analysis of a pure sample over the course of 30 days indeed revealed a steady conversion (t_{1/2} is about 12 days) from the cyclopropenimine 5 to a new compound, which the inventors have identified as 13 (FIG. 5).

It is believed that internal deprotonation of the pendant hydroxyl of cyclopropenimine 5 generates the alkoxy cyclopropenium 14, which can then cyclize to the oxazolidine 15. Destructive ring opening to produce vinyl anion 16 followed by proton transfer would then lead to the observed product 13.

Importantly, this decomposition pathway was greatly slowed by storing the cyclopropenimine 5 at −20° C., with a sample still 94% intact after 30 days. Alternatively, it was found that the HCl salt of 5 is indefinitely stable at room temperature, and given that conversion of 5.HCl to 5 requires only a simple wash with aqueous base, the inventors have found it most convenient to store the catalyst as its acid co-salt.

The inventors also investigated the performance of cyclopropenimine 5 for asymmetric catalysis on a preparative scale. Thus, the addition of glycine imine 2 to methyl acrylate was performed to produce 25 g (97% yield, 99% ee) of the product 3 in 8 hours using 2.5 mol % of catalyst 5 (eq. 3).

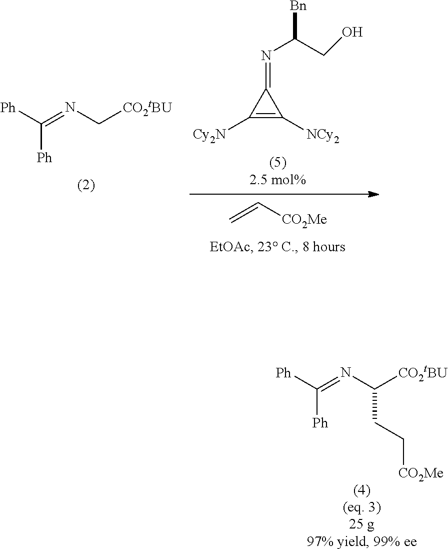

Given that 5 can be easily generated in significant quantities (see eq. 2), it is believed that catalysis with chiral cyclopropenimines should be amenable to relatively large-scale applications.

In summary, the experimental verification of the high basicity of cyclopropenimines provides an important addition to the so-called “superbase” arsenal (Ishikawa, 2009). The exceptional performance of the chiral cyclopropenimine 5 versus related guanidine bases suggests that these new catalysts may enable important developments in the area of enantioselective Brønsted base catalysis. The extraordinary ease of preparation of cyclopropenimines and their amenability to use on multigram scale as we have demonstrated should make cyclopropenimines suitable to a range of applications.

### Example 4

### General Procedures

All reactions were performed using oven-dried glassware under an atmosphere of dry argon, unless otherwise noted. Non-aqueous reagents were transferred by syringe under argon. Organic solutions were concentrated using a Buchi rotary evaporator.

Methylene chloride was dried using a J. C. Meyer solvent purification system. All other solvents and commercial reagents and were used as provided. Tetrachlorocyclopropene (Tobey et al., 1996a & 1966b) and (S)-2-amino-3-phenylpropan-1-ol (Shi et al., 2010) were prepared according to reported procedures. Flash column chromatography was performed employing 32-63 μm silica gel (Dynamic Adsorbents Inc). Thinlayer chromatography (TLC) was performed on silica gel 60 F254 plates (EMD).

¹H and ¹³C NMR were recorded in CDCl₃ (unless otherwise noted) on Bruker DRX-300 and DRX-400 spectrometers as noted. Data for ¹H NMR are reported as follows: chemical shift (δ ppm), multiplicity (s=singlet, br s=broad singlet, d=doublet, t=triplet, q=quartet, qt=quintet, m=multiplet), coupling constant (Hz), integration, and assignment. Data for ¹³C NMR are reported in terms of chemical shift. For neutral cyclopropenimines, cyclopropene carbons were not always observed and cyclohexane carbons appeared broad, likely due to conformational equilibriums; thus all cyclopropenimines have been characterized in their neutral and protonated forms. Optical rotations were measured using a Jasco DIP-1000 digital polarimeter. Lowresolution mass spectrometry (LRMS) was performed on a JEOL JMS-LCmate liquid chromatography spectrometer system using APCl+ ionization technique. HPLC analysis was performed on an Agilent Technologies 1200 series instrument with a Daicel Chiralpak AD-H chiral column (25 cm) using the given conditions (hexanes/isopropanol solvent system).

### Example 5

### Synthesis of Cyclopropenimine 1

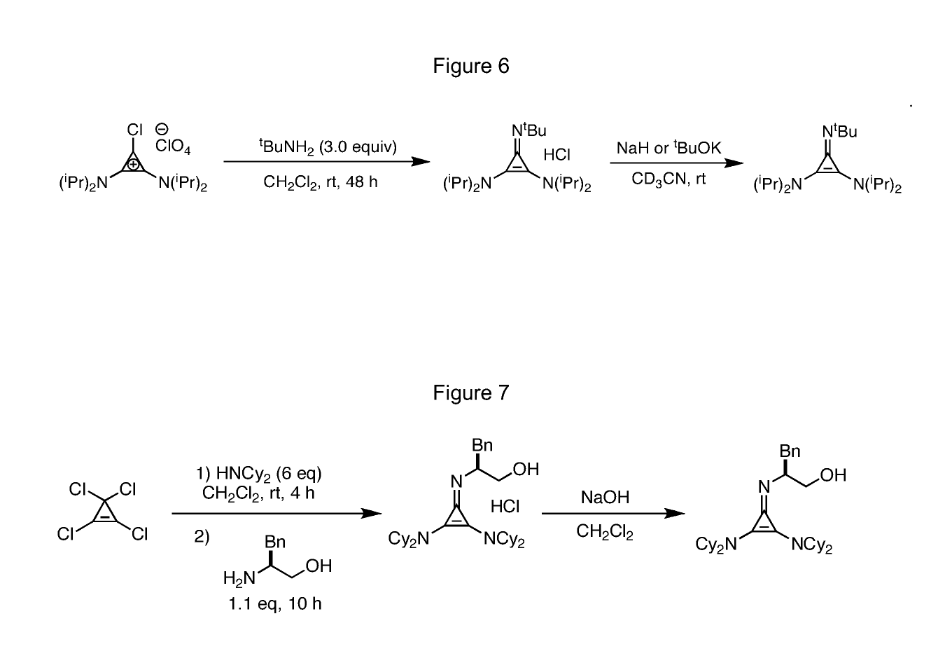

The synthesis scheme for cyclopropenimine compound 1 (shown below) is depicted in FIG. 6.

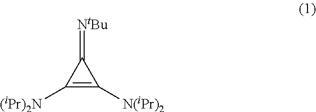

### 3-(tert-butylimino)-N1,N1,N2,N2-tetraisopropylcycloprop-1-ene-1,2-diamine hydrochloride salt (1.HCl)

tert-Butylamine (1.4 mL, 13.25 mmol, 3.0 equiv) was added to a solution of chlorocyclopropenium salt (Yoshida et al., 1971) (1.640 g, 4.42 mmol, 1.0 equiv) in CH₂Cl₂ (50 mL). A white precipitate was observed as the reaction mixture was stirred for 48 hours at room temperature. The crude reaction mixture was washed with 1.0 M HCl (3×30 mL), dried with anhydrous sodium sulfate and concentrated in vacuo to yield pure cyclopropenimine hydrochloride salt as a white solid (1.490 g, 97% yield). The cyclopropenimine salt can be stored at room temperature without noticeable decomposition. ¹H NMR (400 MHz, CD₃CN) δ 5.40 (s, 1H, NH), 4.00 (m, 6.8 Hz, 4H, NCH(CH₃)₂), 1.39 (5, 9H, NC(CH3)3), 1.27 (d, 8 Hz, 24H, NCH(CH₃)2); ¹³C NMR (100 MHz, CD₃CN) δ 117.1, 114.4, 54.0, 51.1, 30.5, 22.3; LRMS (APCl+) m/z=308.31 calculated for C₁₉H₃₇N₃ [M+1]⁺, found 308.47. Neutral cyclopropenimine was prepared in situ in CD3CN to be used for pK_{BH+} estimation. ¹H NMR data was recorded after addition of excess sodium hydride or potassium tert-butoxide to the corresponding cyclopropenimine hydrochloride salt in CD₃CN. ¹H NMR (300 MHz, CD₃CN) δ 3.60 (m, 4H, NCH(CH₃)₂), 1.10-1.40 (m, 33H, NC(CH₃)₃, NCH(CH₃)₂).

### Example 6

### pK_{BH+} Estimation of Cyclopropenimine 1 in CD₃CN

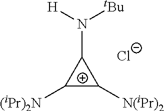

### Using 1,8-Diazabicycloundec-7-ene (DBU)

Cyclopropenimine hydrochloride (18.2 mg, 0.053 mmol, 1.0 equiv) and 1,8-diazabicycloundec-7-ene (7.9 μl, 0.053 mmol, 1.0 equiv) were dissolved in CD₃CN. The ¹H NMR chemical shift of the cyclopropenimine isopropyl groups was used to estimate the equilibrium ratio and equilibrium constant of the reaction. The equilibrium constant was then used, with the known pK_{BH+} of DBU (Kaljurand et al., 2005), to determine the estimated pK_{BH+}. The experiment was repeated in triplicate. A representative ¹H NMR spectrum of the experiment is included.

### Using tert-Butylimino-tri(pyrrolidino)phosphorane (BTPP)

Cyclopropenimine hydrochloride (13.8 mg, 0.04 mmol, 1.0 equiv) and tert-Butylimino-tri(pyrrolidino)phosphorane (12.5 mg, 0.04 mmol, 1.0 equiv) were dissolved in CD₃CN. The ¹H NMR chemical shift of the cyclopropenimine isopropyl groups was used to estimate the equilibrium ratio and equilibrium constant of the reaction. The equilibrium constant was then used, with the known pK_{BH+} of BTPP (Kaljurand et al., 2005), to determine the estimated pK_{BH+}. The experiment was repeated in triplicate. The results are shown below in Table 5.

TABLE 5

Estimated
pK_{BH+}
(CD₃CN)

| | DBU | BTPP |
|---|---|---|
| Trial 1 | 26.75 | 27.00 |
| Trial 2 | 26.74 | 26.85 |
| Trial 3 | 26.74 | 27.03 |
| Average | 26.74 | 26.96 |

### Example 7

### Synthesis of Cyclopropenimine 5

The synthesis scheme for cyclopropenimine compound 5 is depicted in FIG. 7.

### (S)-2-(2,3-bis(dicyclohexylamino)cycloallylamino)-3-phenylpropan-1-ol hydrochloride salt (5.HCl)

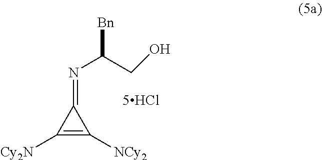

Dicyclohexylamine (93.3 mL, 469.0 mmol, 6.0 equiv) was slowly added to a solution of tetrachlorocyclopropene (9.59 mL, 78.16 mmol, 1.0 equiv) in CH₂Cl₂ (800 mL) in a 3-liter round bottom flask. A white precipitate formed as the reaction mixture was stirred for a further four hours at room temperature. Next, (S)-2-amino-3-phenylpropan-1-ol (13.0 g, 86.0 mmol, 1.1 equiv) was added in one portion and the reaction mixture was stirred for an additional ten hours. The crude reaction mixture was filtered through a celite plug, then washed with 1.0 M HCl (3×400 mL), dried with anhydrous sodium sulfate and concentrated in vacuo to yield pure cyclopropenimine hydrochloride salt as a white solid (45.0 g, 99% yield). The cyclopropenimine salt can be stored at room temperature without noticeable decomposition. ¹H NMR (400 MHz, CDCl₃) δ 7.35 (d, 9.2 Hz, 1H, NH), 7.0-7.15 (m, 5H, ArH), 5.17 (t, 5.6 Hz, ¹H, —OH), 3.60-3.85 (m, 3H, NCHBnCH₂OH), 3.10 (m, 4H, NCyH), 2.80-3.00 (m, 2H, —CH₂Ph), 1.00-1.70 (m, 40H, CyH). 13C NMR (100 MHz, CDCl₃) δ 137.8, 129.2, 128.0, 126.2, 116.5, 114.5, 63.7, 61.3, 59.1, 38.4, 32.1, 31.9, 25.4, 24.4. X-Ray quality crystals were obtained by slow condensation of hexanes into a benzene solution of cyclopropenimine hydrochloride salt.

### (S)-2-(2,3-bis(dicyclohexylamino)cycloallylamino)-3-phenylpropan-1-ol (5)

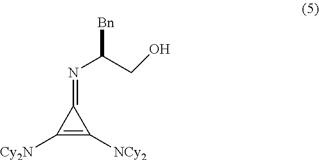

Cyclopropenimine was prepared and stored in a freezer on a weekly basis. Pure cyclopropenimine was quantitatively obtained by dissolving the corresponding hydrochloride salt in CH₂Cl₂ and washing the solution with 1.0 M NaOH (3×), drying with anhydrous sodium sulfate and concentrating in vacuo. The cyclopropenimine is obtained as an off-white solid. ¹H NMR (400 MHz, CDCl₃) δ 7.10-7.25 (m, 5H, ArH), 3.79 (m, ¹H, NCHBnCH₂OH), 3.40-3.50 (m, 2H, NCHBnCH₂OH), 3.00-3.10 (m, 4H, NCyH), 2.70-2.85 (m, 2H, —CH₂Ph), 1.00-1.90 (m, 40H, CyH). 13C NMR (100 MHz, CDCl₃) δ 140.5, 129.7, 129.4, 127.8, 125.5, 65.1, 61.7, 58.3, 41.7, 34.4, 33.1, 32.8, 32.6, 26.3, 26.1, 25.3, 25.2; [α]²⁰_{D}=−0.8 (1.0 c, CHCl₃); LRMS (APCl+) m/z=546.43 calculated for C₃₆H₅₅N₃O [M+1]⁺, found 546.98.

### Example 8

### Synthesis of Cyclopropenimine 7

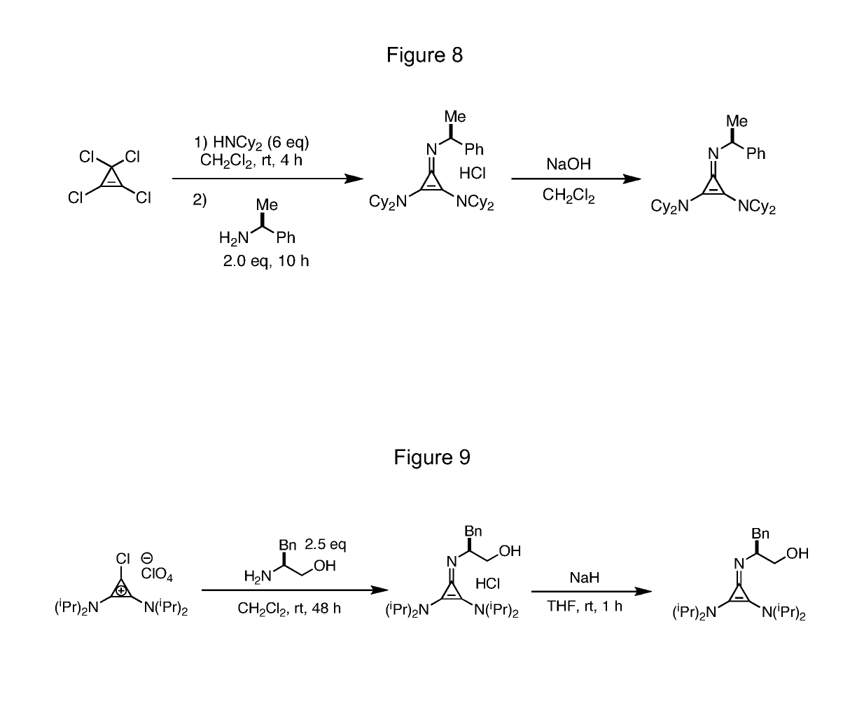

The synthesis scheme for cyclopropenimine compound 7 is depicted in FIG. 8.

### (S)—N1,N1,N2,N2-tetracyclohexyl-3-(1-phenylethylimino)cycloprop-1-ene-1,2-diamine hydrochloride salt (7.HCl)

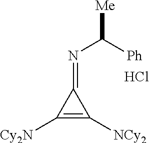

Dicyclohexylamine (1.27 mL, 6.42 mmol, 6.0 equiv) was slowly added to a solution of tetrachlorocyclopropene (0.13 mL, 1.07 mmol, 1.0 equiv) in CH₂Cl₂ (20 mL) at room temperature. A white precipitate formed as the reaction mixture was stirred for a further four hours. Next, (S)-1-phenylethanamine (0.28 mL, 2.14 mmol, 2.0 equiv) was added in one portion and the reaction mixture was stirred overnight. The crude reaction mixture was filtered through a celite plug, then washed with 1.0 M HCl (3×20 mL), dried with anhydrous sodium sulfate and concentrated in vacuo to yield pure cyclopropenimine hydrochloride salt as a white solid (567 mg, 96% yield). The cyclopropenimine salt can be stored at room temperature without noticeable decomposition. ¹H NMR (400 MHz, CDCl₃) δ 8.85 (d, 8.0 Hz, 1H, NH), 7.45 (d, 7.6 Hz, 2H, ArH), 7.35 (t, 7.6 Hz, 2H, ArH), 7.20 (t, 7.2 Hz, 2H, ArH), 4.90 (qt, 7.2 Hz, 1H, NCHMePh), 3.25-3.35 (m, 4H, NCyH), 1.20-1.90 (m, 43H, CyH, CH₃); 13C NMR (100 MHz, CDCl₃) δ 143.6, 128.7, 127.0, 125.8, 116.3, 115.7, 59.4, 57.0, 32.2, 32.0, 25.5, 25.5, 24.5, 24.1.

### (S)—N1,N1,N2,N2-tetracyclohexyl-3-(1-phenylethylimino)cycloprop-1-ene-1,2-diamine (7)

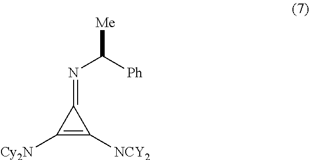

Pure cyclopropenimine was quantitatively obtained by dissolving the corresponding hydrochloride salt in CH₂Cl₂ and washing the solution with 1.0 M NaOH (3×), drying with anhydrous sodium sulfate and concentrating in vacuo. The cyclopropenimine is obtained as an off-white solid. ¹H NMR (400 MHz, CDCl₃) δ 7.52 (d, 7.2 Hz, 2H, ArH), 7.25 (t, 7.6 Hz, 2H, ArH), 7.10 (t, 7.2 Hz, 1H, ArH), 4.80 (q, 6.4 Hz, 1H, NCHMePh), 3.10 (br s, 4H, NCyH), 1.10-1.90 (m, 40H, CyH), 1.45 (d, 6.4 Hz, 3H, CH₃); ¹³C NMR (100 MHz, CDCl₃) δ 127.8, 126.7, 125.3, 59.6, 58.4, 33.2, 32.9, 28.7, 26.4, 25.4; [α]₂₀^{D}=−0.8 (1.0 c, CHCl₃); LRMS (APCl+) m/z=516.43 calculated for C₃₅H₅₃N₃ [M+1]⁺, found 517.07.

### Example 9

### Synthesis of Cyclopropenimine 8

The synthesis scheme for cyclopropenimine compound 8 is depicted in FIG. 9.

### (S)-2-(2,3-bis(diisopropylamino)cycloallylamino)-3-phenylpropan-1-ol hydrochloride salt (8.HCl)

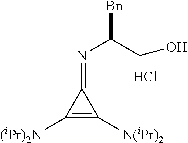

(S)-2-amino-3-phenylpropan-1-ol (400 mg, 2.65 mmol, 2.5 equiv) was added to a solution of chlorocyclopropenium salt (390 mg, 1.06 mmol, 1.0 equiv) in CH₂Cl₂ (10 mL) (Yoshida, 1971). A white precipitate was observed as the reaction mixture was stirred for 48 hours at room temperature.

The crude reaction mixture was washed with 1.0 M HCl (3×10 mL), dried with anhydrous sodium sulfate and concentrated in vacuo to yield pure cyclopropenimine hydrochloride salt as a pale yellow solid (440 mg, 98% yield). The cyclopropenimine salt can be stored at room temperature without noticeable decomposition. ¹H NMR (400 MHz, CDCl₃) δ 7.10-7.25 (m, 5H, ArH), 6.30 (d, 9.6 Hz, NH), 3.60-4.0 (m, 8H, NCHBnCH₂OH, NCH(CH₃)₂), 1.20 (m, 24H, NCH(CH₃)₂); ¹³C NMR (100 MHz, CDCl₃) δ 137.9, 129.5, 128.5, 126.6, 116.3, 113.7, 64.6, 62.4, 50.5, 38.1, 22.1, 22.0.

### (S)-2-(2,3-bis(diisopropylamino)cycloallylamino)-3-phenylpropan-1-ol (8)

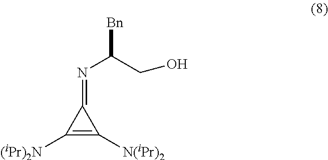

Sodium hydride (28 mg, 60% in mineral oil, 0.711 mmol, 3.0 equiv) was washed with pentanes (3×2 mL) then added to acetonitrile (2.0 mL). Next, a solution of the corresponding cyclopropenimine hydrochloride salt (100 mg, 0.237 mmol, 1.0 equiv) in acetonitrile (1.0 mL) was added and the reaction mixture was stirred for 15 minutes at room temperature. The reaction solution was filtered through celite and concentrated in vacuo yield pure cyclopropenimine as a white solid (50 mg, 55% yield). ¹H NMR (400 MHz, CD3CN) δ 7.10-7.30 (m, 5H, ArH), 3.60-3.75 (m, 5H, NCH(CH₃)₂, NCHBnCH2OH), 3.20-3.40 (m, 2H, NCHBnCH₂OH), 2.60-2.80 (m, 2H, NCHCH₂Ph), 1.20 (d, 6.8 Hz, 24H, NCH(CH₃)₂); ¹³C NMR (100 MHz, CD3CN) δ 141.5, 130.5, 128.8, 128.3, 126.5, 66.6, 64.6, 50.1, 42.3, 22.6, 22.5; [α]²⁰_{D}=−1.0 (2.0 c, CHCl₃); LRMS (APCl+) m/z=386.32 calculated for C₂₄H₃₉N₃O [M+1]⁺, found 386.31.

### Example 10

### Cyclopropenimine 5 Rearrangement

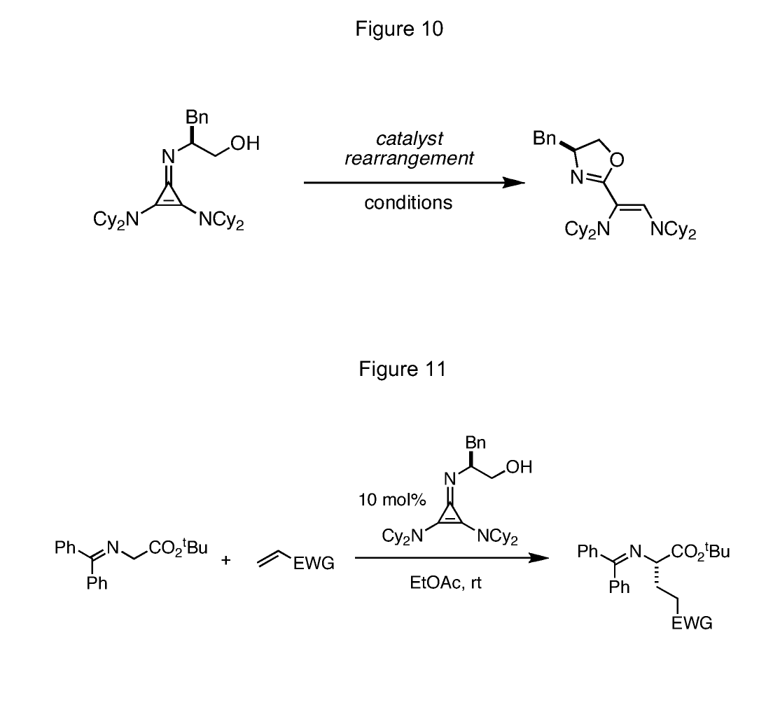

The rearrangement for cyclopropenimine compound 5 is depicted in FIG. 10.

### Rearrangement Rate Study:

Freshly obtained cyclopropenimine 5 was placed in a vial (250 mg per vial) and stored at room temperature and in a −20° C. freezer. Freshly isolated cyclopropenimine hydrochloride salt (250 mg of 5.HCl) was also stored at room temperature. At each time point, a small amount of the solid was removed and its ¹H NMR (CDCl₃) was used to measure the ratio of cyclopropenimine to the rearranged product. The percent purity of starting cyclopropenimine are as shown in Table 6 below.

TABLE 6

| | Cyclopropenimine | | |
|---|---|---|---|
| | purity (%) | | |
| Time (days) | rt | −20° C. | HCl salt |
| 0 | 100 | 100 | 100 |
| 2 | 78 | 100 | 100 |
| 9 | 57 | 98 | 100 |
| 16 | 39 | 96 | 100 |
| 23 | 25 | 96 | 100 |
| 30 | 5 | 94 | 100 |

Rearranged product was purified by passing crude rearranged material through a silica gel plug treated with triethylamine in a 1% triethylamine/ethyl acetate mixture. Rearranged product was obtained as a white solid. ¹H NMR (300 MHz, CDCl₃) δ 7.10-7.30 (m, 5H, ArH), 6.95 (s, 1H, C═CH), 5.50 (br s, 1H), 4.25 (m, 1H), 3.85 (d, 6.9 Hz, —CH₂O), 2.70-3.20 (m, 4H), 3.50 (m, 1H), 1.00-2.00 (m, 40H, CyH); ¹³C NMR (75 MHz, CDCl₃) δ 168.3, 139.2, 139.1, 129.4, 128.3, 126.0, 100.3, 69.3, 67.6, 57.5, 57.0, 54.4, 42.1, 34.5, 33.5, 33.2, 31.1, 26.8, 26.2, 25.7; [α]₂₀^{D}=7.7 (1.0 c, CHCl₃); LRMS (APCl+) m/z=546.44 calculated for C₃₆H₅₅N₃O [M+1]⁺, found 547.28.

### Example 11

### General Procedure for Glycinate-Michael Additions

The synthesis scheme for glycinate-Michael Additions is depicted in FIG. 11.

Specifically, cyclopropenimine 5 (3.6 mg, 0.00677 mmol, 0.1 equiv) and tert-Butyl glycinate benzophenone Schiff base (20 mg, 0.0677 mmol, 1.0 equiv) were dissolved in ethyl acetate (0.2 mL). Michael acceptor (0.203 mmol, 3.0 equiv) was then added to the vial and the reaction solution was stirred at room temperature. Upon complete consumption of starting material, monitored by ¹H NMR, the reaction solution was concentrated and the crude material subjected to silica gel column chromatography (Et₂O/Hexanes eluent). Table 2 above shows the substrate scope of Michael acceptors.

### (S)-1-tert-butyl-5-methyl 2-(diphenylmethyleneamino)pentanedioate

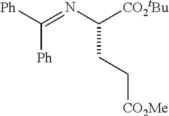 [SMILES: COC(=O)CC[C@H](N=C(c1ccccc1)c1ccccc1)C(=O)OC(C)(C)C]

(S)-1-tert-butyl-5-methyl 2-(diphenylmethyleneamino)pentanedioate (Ma et al., 2011) is also shown in Table 2 above, under entry 1.

(S)-1-tert-butyl-5-methyl 2-(diphenylmethyleneamino)pentanedioate was isolated as a colorless oil (26 mg, 100% yield). ¹H NMR (400 MHz, CDCl₃) δ 7.64 (d, 7.2 Hz, 2H, ArH), 7.14-7.45 (m, 8H, ArH), 3.97 (t, 6.4 Hz, HCCO₂ tBu), 3.59 (s, 3H, CO₂CH₃), 2.35-2.45 (m, 2H, —CH₂—), 2.15-2.25 (m, 2H, —CH₂—), 1.44 (s, 9H, C(CH₃)₃); ¹³C NMR (100 MHz, CDCl₃) δ 173.7, 170.9, 170.8, 139.6, 136.6, 130.4, 128.9, 128.7, 128.6, 128.1, 127.9, 81.3, 64.9, 51.6, 30.6, 28.8, 28.2. HPLC analysis: Chiralpak AD-H (Hex/IPA=97/3, 1.0 mL/min, 254 nm, 23° C.), 6.1 min (minor), 6.7 min (major), 98% ee.

### Large-Scale:

The general procedure was followed using cyclopropenimine 5 (0.924 g, 1.693 mmol, 0.025 equiv), tert-Butyl glycinate benzophenone Schiff base (20.0 g, 67.7 mmol, 1.0 equiv) and methyl acrylate (18.4 mL, 203.1 mmol, 3.0 equiv) dissolved in ethyl acetate (200 mL). The product was isolated as a colorless oil (25.03 g, 97% yield). HPLC analysis: Chiralpak AD-H (Hex/IPA=97/3, 1.0 mL/min, 254 nm, 23° C.), 6.1 min (minor), 6.7 min (major), 99% ee.

### (S)-1-tert-butyl-5-butyl 2-(diphenylmethyleneamino)pentanedioate

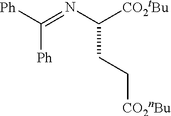

(S)-1-tert-butyl-5-butyl 2-(diphenylmethyleneamino)pentanedioate (Ohshima et al., 2004) is also listed in Table 2 above, under entry 2.

(S)-1-tert-butyl-5-butyl 2-(diphenylmethyleneamino)pentanedioate was isolated as a colorless oil (29 mg, 100% yield). ¹H NMR (400 MHz, CDCl₃) δ 7.64 (m, 2H, ArH), 7.30-7.45 (m, 6H, ArH), 7.15-7.20 (m, 2H, ArH), 3.90-4.00 (m, 3H, HCCO₂ tBu, CO₂CH₂), 2.35 (m, 2H, —CH₂—), 2.20 (m, 2H, —CH₂), 1.55 (m, 2H, —CH₂—), 1.44 (s, 9H, C(CH₃)₃, 1.35 (m, 2H, —CH₂—), 0.90 (t, 7.2 Hz, —CH₃); 13C NMR (100 MHz, CDCl₃) δ 173.4, 170.9, 170.8, 139.7, 136.7, 130.4, 128.9, 128.7, 128.6, 128.1, 127.9, 81.3, 65.0, 64.4, 30.9, 30.8, 28.9, 28.2, 19.2, 13.8. HPLC analysis: Chiralpak AD-H (Hex/IPA=98/2, 1.0 mL/min, 254 nm, 23° C.), 6.7 min (minor), 7.4 min (major), 98% ee.

### (S)-di-tert-butyl 2-(diphenylmethyleneamino)pentanedioate

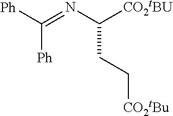

(S)-di-tert-butyl 2-(diphenylmethyleneamino)pentanedioate (Saito et al., 2007) is also listed in Table 2 above, under entry 3.

(S)-d i-tert-butyl 2-(diphenylmethyleneamino)pentanedioate was isolated as a colorless oil (28 mg, 98% yield). ¹H NMR (400 MHz, CDCl₃) δ 7.65 (d, 7.2 Hz, ArH), 7.30-7.50 (m, 6H, ArH), 7.20 (m, 2H, ArH), 3.95 (t, 5.6 Hz, ¹H, HCCO₂ tBu), 2.15-2.30 (m, 4H, —CH2CH2-), 1.44 (s, 9H, C(CH3)3), 1.39 (s, 9H, C(CH3)3); 13C NMR (100 MHz, CDCl₃) δ 172.6, 171.0, 170.7, 139.7 136.7, 130.4, 128.9, 128.7, 128.6, 128.1, 128.0, 81.2, 80.3, 65.1, 32.1, 29.0, 28.2. HPLC analysis: Chiralpak AD-H (Hex/IPA=98/2, 0.6 mL/min, 254 nm, 23° C.), 9.0 min (minor), 9.6 min (major), 99% ee.

### (S)-5-benzyl-1-tert-butyl 2-(diphenylmethyleneamino)pentanedioate

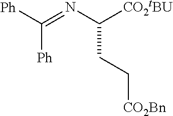

(S)-5-benzyl-1-tert-butyl 2-(diphenylmethyleneamino)pentanedioate (Ma et al., 2011) is also listed in Table 2 above, under entry 4.

(S)-5-benzyl-1-tert-butyl 2-(diphenylmethyleneamino)pentanedioate was isolated as a colorless oil (30 mg, 97% yield). ¹H NMR (400 MHz, CDCl₃) δ 7.63 (m, 2H, ArH), 7.30-7.45 (m, 1¹H, ArH), 7.15 (m, 2H, ArH), 5.03 (s, 2H, CO₂CH₂Ph), 3.97 (dd, 7.2 Hz, 5.2 Hz, ¹H, HCCO₂ ^{t}Bu), 2.44 (m, 2H, —CH₂—), 2.35 (m, 2H, —CH₂—), 1.43 (s, 9H, C(CH₃)₃); ¹³C NMR (100 MHz, CDCl₃) δ 173.1, 170.9, 170.8, 139.6, 136.6, 136.1, 130.5, 129.0, 128.7, 128.6, 128.6, 128.3, 128.1, 127.9, 81.3, 66.3, 65.0, 30.9, 28.8, 28.2. HPLC analysis: Chiralpak AD-H (Hex/IPA=98/2, 1.0 mL/min, 254 nm, 23° C.), 11.1 min (minor), 12.8 min (major), 98% ee.

### (S)-tert-butyl 2-(diphenylmethyleneamino)-5-oxohexanoate

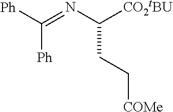

(S)-tert-butyl 2-(diphenylmethyleneamino)-5-oxohexanoate (Ma et al., 2011) is also listed in Table 2 above, under entry 5.

(S)-tert-butyl 2-(diphenylmethyleneamino)-5-oxohexanoate was isolated as a colorless oil (24 mg, 97% yield). ¹H NMR (400 MHz, CDCl₃) δ 7.63 (m, 2H, ArH), 7.30-7.50 (m, 6H, ArH), 7.15 (m, 2H, ArH), 3.96 (t, 6.4 Hz, 1H, HCCO₂ ^{t}Bu), 2.45-2.60 (m, 2H, —CH₂—), 2.15 (m, 2H, —CH₂—), 2.12 (s, 3H, —COCH₃), 1.43 (s, 9H, C(CH₃)₃); ¹³C NMR (100 MHz, CDCl₃) δ 208.4, 171.1, 170.6, 139.6, 136.6, 130.4, 128.9, 128.7, 128.6, 128.2, 127.9, 81.3, 64.8, 40.0, 30.0, 28.2, 27.9. HPLC analysis: Chiralpak AD-H (Hex/IPA=97/3, 1.0 mL/min, 254 nm, 23° C.), 7.2 min (minor), 8.1 min (major), 95% ee.

### (S)-tert-butyl 4-cyano-2-(diphenylmethyleneamino)butanoate

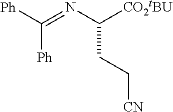

(S)-tert-butyl 4-cyano-2-(diphenylmethyleneamino)butanoate (Zhang et al., 2000) is also listed in Table 2 above, under entry 6.

(S)-tert-butyl 4-cyano-2-(diphenylmethyleneamino)butanoate was obtained as a pale yellow oil (23 mg, 98% yield). ¹H NMR (400 MHz, CDCl₃) δ 7.66 (m, 2H, ArH), 7.30-7.50 (m, 6H, ArH), 7.20 (m, 2H, ArH), 4.05 (m, 1H, HCCO₂ ^{t}Bu), 2.20-2.50 (m, 4H, —CH₂CH₂—), 1.44 (s, 9H, C(CH₃)₃); ¹³C NMR (100 MHz, CDCl₃) δ 172.1, 170.0, 139.2, 136.1, 130.7, 129.0, 128.9, 128.7, 128.2, 127.5, 119.5, 81.9, 63.8, 29.5, 28.1, 13.9. HPLC analysis: Chiralpak AD-H (Hex/IPA=98/2, 1.0 mL/min, 254 nm, 23° C.), 9.3 min (minor), 11.7 min (major), 77% ee.

### (S)-tert-butyl 2-(diphenylmethyleneamino)-4-(phenylsulfonyl)butanoate

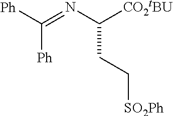

(S)-tert-butyl 2-(diphenylmethyleneamino)-4-(phenylsulfonyl)butanoate (Tsubogo et al., 2008) is also listed in Table 2 above, under entry 7.

(S)-tert-butyl 2-(diphenylmethyleneamino)-4-(phenylsulfonyl)butanoate was isolated with phenyl vinyl sulfone starting material (88% yield based on benzyl ether standard). ¹H NMR (400 MHz, CDCl₃) δ 7.90 (m, 2H, ArH), 7.50-7.70 (m, 5H, ArH), 7.30-7.45 (m, 6H, ArH), 7.10 (m, 2H, ArH), 4.00 (m, 1H, HCCO₂ ^{t}Bu), 3.10-3.35 (m, 2H, CH₂SO₂Ph), 2.10-2.30 (m, 2H, CH₂CH₂SO₂Ph), 1.37 (s, 9H, C(CH₃)₃); ¹³C NMR (100 MHz, CDCl₃) δ 171.5, 169.9, 139.1, 139.0, 136.1, 130.7, 129.4, 128.9, 128.9, 128.7, 128.2, 128.1, 127.7, 81.8, 63.6, 52.9, 28.0, 27.1. HPLC analysis: Chiralpak AD-H (Hex/IPA=95/5, 1.0 mL/min, 254 nm, 23° C.), 12.9 min (minor), 16.8 min (major), 41% ee. tert-butyl 2-(diphenylmethyleneamino)-5-oxo-3,5-diphenylpentanoate

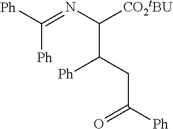

tert-butyl 2-(diphenylmethyleneamino)-5-oxo-3,5-diphenylpentanoate (Ma et al., 2011) is listed in Table 2 above, under entry 8.

tert-butyl 2-(diphenylmethyleneamino)-5-oxo-3,5-diphenylpentanoate was isolated as a white solid (33 mg, 97% yield). A 6:1 diastereomeric ratio was determined by ¹H NMR of the crude reaction mixture. ¹H NMR (400 MHz, CDCl₃) δ 8.00 (d, 7.2 Hz, 2H, ArH), 7.70 (d, 7.2 Hz, 2H, ArH), 7.30-7.60 (m, 9H, ArH), 7.10-7.20 (m, 5H, ArH), 6.70 (m, 2H, ArH), 4.20 (m, 2H, NCHCO₂ ^{t}Bu, CHPh), 3.60-3.80 (m, 2H, CH₂COPh), 1.33 (s, 9H, C(CH₃)₃); ¹³C NMR (100 MHz, CDCl₃) δ 198.8, 171.2, 170.1, 141.5, 139.5, 137.3, 136.4, 132.9, 130.4, 129.0, 128.7, 128.6, 128.5, 128.3, 128.3, 128.2, 128.1, 127.6, 126.7, 81.4, 44.9, 40.1, 28.0. HPLC analysis: Chiralpak AD-H (Hex/IPA=95/5, 1.0 mL/min, 254 nm, 23° C.), 8.8 min (minor), 11.2 min (major), 95% ee.

### Example 12

### Synthesis of Compound 42

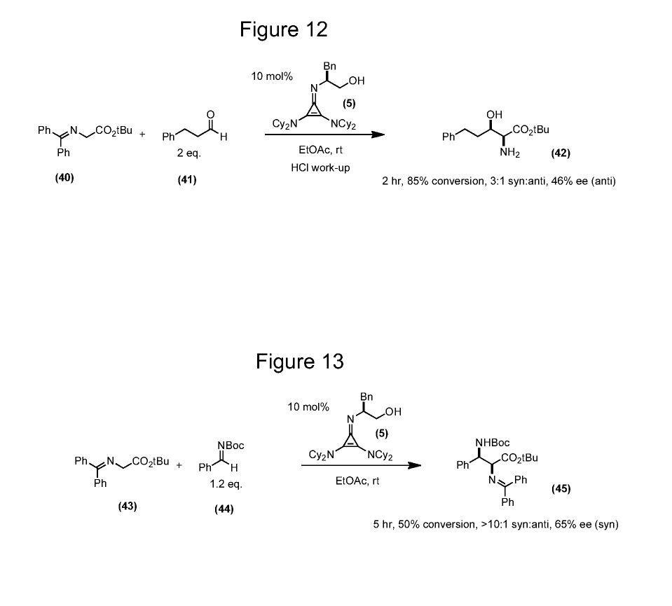

The synthesis scheme for compound 42 is shown in FIG. 12. Compound 40 and compound 41 (2 equiv) were reacted in the presence of cyclopropenimine 5 (10 mol %) with ethyl acetate as the solvent. The reaction was carried out at room temperature for 2 hours, followed by HCl work up. The reaction proceeded with 3:1 ratio of syn:anti. The yield was 85%, 46% ee (anti).

### Example 13

### Synthesis of Compound 45

The synthesis scheme for compound 45 is shown in FIG. 13. Compound 43 and compound 44 (1.2 equiv) were reacted in the presence of cyclopropenimine 5 (10 mol %) with ethyl acetate as the solvent. The reaction was carried out at room temperature for 5 hours. The reaction proceeded with greater than 10:1 ratio of syn:anti. The yield was 50%, 65% ee (syn).

### Example 14

### Synthesis of Compound 48

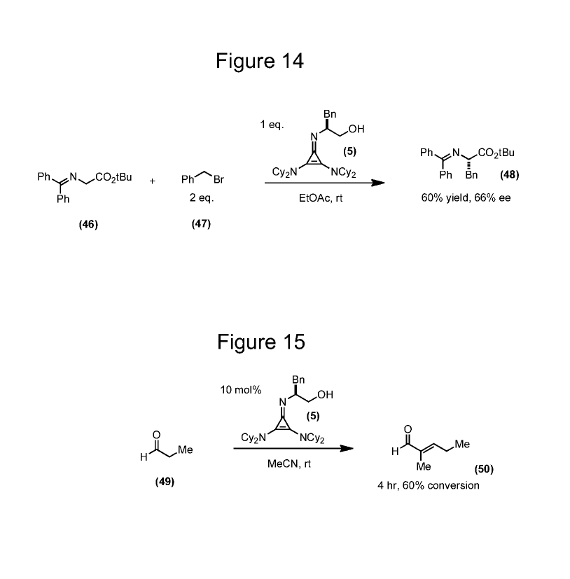

The synthesis scheme for compound 48 is shown in FIG. 14. Compound 46 and compound 47 (2 equiv) were reacted in the presence of cyclopropenimine 5 (1 equiv) with ethyl acetate as the solvent. The reaction was carried out at room temperature. The yield was 60%, 66% ee.

### Example 15

### Synthesis of Compound 50

The synthesis scheme for compound 50 is shown in FIG. 15. Compound 49 was reacted in the presence of cyclopropenimine 5 (10 mol %) with acetonitril as the solvent. The reaction was carried out at room temperature for 4 hours. The yield was 60%.

### Example 16

### General Procedures for Mannich Reactions

In general, the Mannich reaction set forth in Example 2 above was carried out as follows. Glycine imine was dissolved in toluene, followed by the addition of cyclopropenimine catalyst and activated molecular sieves. Once all materials were dissolved, N-Boc-aldimine was added, and the reaction was stirred at room temperature open to air. Reaction progress was monitored by HNMR spectroscopy, and the desired product was purified from the crude reaction material via flash column chromatography.

The cyclopropenimine catalyst was prepared according to one of the procedures set forth in the Examples above. Glycine imines were synthesized according to Wu et al. (1982). N-Boc-aldimines were synthesized according to Wenzel et al. (2002) and Song et al. (2006). All other starting materials were purchased from Sigma Aldrich (St. Louis, Mo.).

### DOCUMENTS

Breslow, R. J. Am. Chem. Soc. 1957, 79, 5318.
Breslow, R.; Yuan, C. J. Am. Chem. Soc. 1958, 80, 5991.
Bruns, H.; Patil, M.; Carreras, J.; Vazquez, A.; Thiel, W.; Goddard, R.; Alcarazo, M. Angew. Chem. Int. Ed. 2010, 49, 3680.
Christoffers, J. A. Baro and T. Werner, Adv. Synth. Catal., 2004, 346, 143-151
D'yakonov, I. A.; Kostikov, R. R. Russ. Chem. Rev. 1967, 36, 557-563.
Duthaler, R. O. Angew. Chem., Int. Ed., 2003, 42, 975-978
Eicher, T.; Graf, R.; Konzmann, H. Synthesis 1987, 887-892.
Eicher, T.; Lerch, D. Tetrahedron Lett. 1980, 21, 3751-3754.
Fu, X.; Tan, C.-H. Chem. Commun. 2011, 47, 8210-8222.
Gattin, Z.; Kovacevic, B.; Maksic, Z. B. Eur. J. Org. Chem. 2005, 3206-3213.
Greck, C. B. Drovillat and C. Thomassigny, Eur. J. Org. Chem., 2004, 1377-1385.
Hashimoto, T.; Maruoka, K. Chem. Rev. 2007, 107, 5656-5682.
Hernandez-Toribio, J.; Arrayas, R. G.; Carretero, J. C. Chem. Eur. J., 2010, 16, 1153-1157.
Ibrain, H. and A. Togni, Chem. Commun., 2004, 1147-1155;
Ishikawa, T.; Araki, Y.; Kumamoto, T.; Seki, H.; Fukuda, K.; Isobe, T. Chem. Commun. 2001, 245-246.
Isobe, T.; Fukuda, K.; Araki, Y.; Ishikawa, T. Chem. Commun. 2001, 243-244;
Isobe, T.; Fukuda, K.; Ishikawa, T. J. Org. Chem. 2000a, 65, 7770-7773.
Isobe, T.; Fukuda, K.; Ishikawa, T. J. Org. Chem. 2000b, 65, 7774-7778;
Isobe, T.; Fukuda, K.; Ishikawa, T. Tetrahedron: Asymmetry 1998, 9, 1729-1735.
Isobe, T.; Fukuda, K.; Yamaguchi, K.; Seki, H.; Tokunaga, T.; Ishikawa, T. J. Org. Chem. 2000c, 65, 7779-7785.
Janey, J. M. Angew. Chem., Int. Ed., 2005, 117, 4364-4372.
Kaljurand, I.; Kütt, A.; Sooväli, L.; Rodima, T.; Mäemets, V.; Leito, I.; Koppel, I. A. J. Org. Chem. 2005, 70, 1019-1028.
Kitani, Y.; Kumamoto, T.; Isobe, T.; Fukuda, K.; Ishikawa, T. Adv. Synth. Catal. 2005, 347, 1653-1658;
Kobayashi et. al. Angew. Chem. Int. Ed. 2008, 47, 5613-5615.
Komatsu, K.; Kitagawa, T. Chem. Rev. 2003, 103, 1371-1427.
Krebs, A. W. Angew. Chem. Int. Ed. 1965, 4, 10-22.
Krebs, A.; Gunter, A.; Versteylen, S.; Schulz, S. Tetrahedron Lett. 1984, 25, 2333-2336.
Kumamoto, T.; Ebine, K.; Endo, M.; Araki, Y.; Fushimi, Y.; Miyamoto, I.; Ishikawa, T.; Isobe, T.; Fukuda, K. Heterocycles 2005, 66, 1454-1455;
Leow, D.; Tan, C.-H. Synlett 2010, 1589-1605.
Ma, T.; Fu, X.; Kee, W. K.; Zong, L.; Pan, Y.; Huang, K. W.; Tan, C. H. J. Am. Chem. Soc. 2011, 133, 2828-2831.
Maksic, Z. B.; Kovacevic, B. J. Phys. Chem. A 1999, 103, 6678-6684.
Marcelli, T.; Hiemstra, H. Synthesis 2010, 1229-1279.
Merino, P. and T. Tejero, Angew. Chem., Int. Ed., 2004, 43, 2995-2997
Milbrath, D. S.; Verkade, J. G. J. Am. Chem. Soc. 1977, 99, 6607-6613.
O'Donnell, M. J. Acc. Chem. Res. 2004, 37, 506-517.
Oestrech, M. Angew. Chem., Int. Ed., 2005, 44, 2324-2327;
Ohshima, T.; Shibuguchi, T.; Fukuta, Y.; Shibasaki, M. Tetrahedron, 2004, 60, 7743-7754.
Palomo, C.; Oiarbide, M.; Lopez, R. Chem. Soc. Rev. 2009, 38, 632-653.
Paquette, L. A.; Barton, T. J.; Horton, N. Tetrahedron Lett. 1967, 8, 5039-5042.
Pihko, P. M. Angew. Chem., Int. Ed., 2006, 45, 544-547;
Pilli, R. A.; Rodrigues, J. A. R.; Kascheres, A. J. Org. Chem. 1983, 48, 1084-1091.
Plietker, B. Tetrahedron: Asymmetry, 2005, 16, 3453-3459
Prakash, G. K. S, and P. Beller, Angew. Chem., Int. Ed., 2006, 45, 2172-2174.
Ryoda, A.; Yajima, N.; Haga, T.; Kumamoto, T.; Nakanishi, W.; Kawahata, M.; Yamaguchi, K.; Ishikawa, T. J. Org. Chem. 2008, 73, 133-141.
Saito, N.; Ryoda, A.; Nakanishi, W.; Kumamoto, T.; Ishikawa, T. Eur. J. Org. Chem. 2008, 2759-2766.
Saito, S.; Tsubogo, T.; Kobayashi, S. J. Am. Chem. Soc. 2007, 129, 5364-5365.
Schoeller, W. W.; Frey, G. D.; Bertrand, G. Chem. Eur. J. 2008, 14, 4711-4718.
Schwesinger, R.; Schlemper, H. Angew. Chem. Int. Ed. 1987, 26, 1167-1169.
Shi, L.; Chen, L.; Chen, R.; Chen, L. J. Label Compd. Radiopharm. 2010, 53, 147-151.
Song et al., J. Am. Chem. Soc., 2006, 128 (18), pp 6048-6049
Superbases for Organic Synthesis: Guanidines, Amidines, and Phosphazenes and Related Organocatalysts Ishikawa, T. Ed.; John Wiley and Sons, 2009.
Tobey, S. W.; West, R. J. Am. Chem. Soc. 1966a, 88, 2478-2481.
Tobey, S. W.; West, R. J. Am. Chem. Soc. 1966b, 88, 2481-2488.
Tsubogo, T.; Saito, S.; Seki, K.; Yamashita, Y.; Kobayashi, S. J. Am. Chem. Soc. 2008, 130, 13321-13332.
Weiss, R.; Hertel, M. J. Chem. Soc. Chem. Commun. 1980, 223-224.
Wenzel et al., J. Am. Chem. Soc., 2002, 124 (44), pp 12964-12965.
West, R.; Tobey, S. W. J. Am. Chem. Soc. 1966, 88, 2481-2488.
Wu et al., J. Org. Chem., 1982, 47 (13), pp 2663-2666.
Yoshida, Z.; Tawara, Y. J. Am. Chem. Soc. 1971, 93, 2573-2574.
Yoshida, Z.-i. Topics in Current Chemistry 1973, 40, 47-72.
Zhang, F. Y.; Corey, E. J.; Org. Lett. 2000, 2, 1097-1100.
Zhang, G.; Kumamoto, T.; Heima, T.; Ishikawa, T. Tetrahedron Lett. 2010, 51, 3927-3930.

All documents cited in this application are hereby incorporated by reference as if recited in full herein.

Although illustrative embodiments of the present invention have been described herein, it should be understood that the invention is not limited to those described, and that various other changes or modifications may be made by one skilled in the art without departing from the scope or spirit of the invention.

What is claimed is:

1. A cyclopropenimine Brønsted base catalyst having the structure: 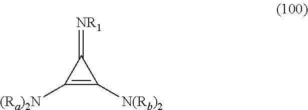
wherein
R₁ is independently selected from the group consisting of aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀cycloalkenyl, C₂₋₁₀alkynyl, halogen, aryloxy, heteroaryloxy, C₂₋₁₀alkoxycarbonyl, C₁₋₁₀alkylthio, C₂₋₁₀alkenylthio, C₂₋₁₀alkynylthio, C₁₋₁₀alkylsulfonyl, C₁₋₁₀alkylsulfinyl aryl-C₁₋₁₀alkyl, heteroaryl-C₁₋₁₀alkyl, aryl-C₁₋₁₀heteroalkyl, heteroaryl-C₁₋₁₀heteroalkyl; wherein the aliphatic or aromatic portions of R₁ are optionally substituted with from 1 to 4 substituents selected from the group consisting of halogen, cyano, nitro, C₁₋₄-alkyl, C₂₋₆alkenyl, C₂₋₆alkynyl, aryl, C₁₋₆alkoxy, C₂₋₆alkenyloxy, C₂₋₆alkynyloxy, aryloxy, C₂₋₆alkoxycarbonyl, C₁₋₆ alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus groups; and
each Rₐ and R_{b} are the same or different and are independently selected from sterically demanding substituents; and
crystalline forms, hydrates, or salts thereof.

2. The cyclopropenimine Brønsted base catalyst according to claim 1, wherein the R₁ is substituted with a hydroxy.

3. The cyclopropenimine Brønsted base catalyst according to claim 1, wherein the sterically demanding substituent is selected from the group consisting of linear, branched, and cyclic alkyl substituents.

4. The cyclopropenimine Brønsted base catalyst according to claim 1 having a structure selected from the group consisting of: 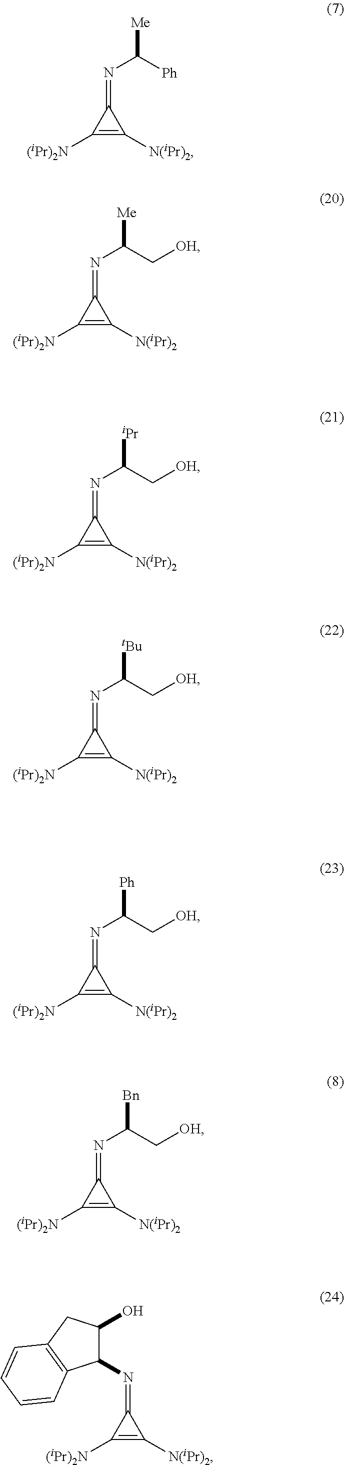 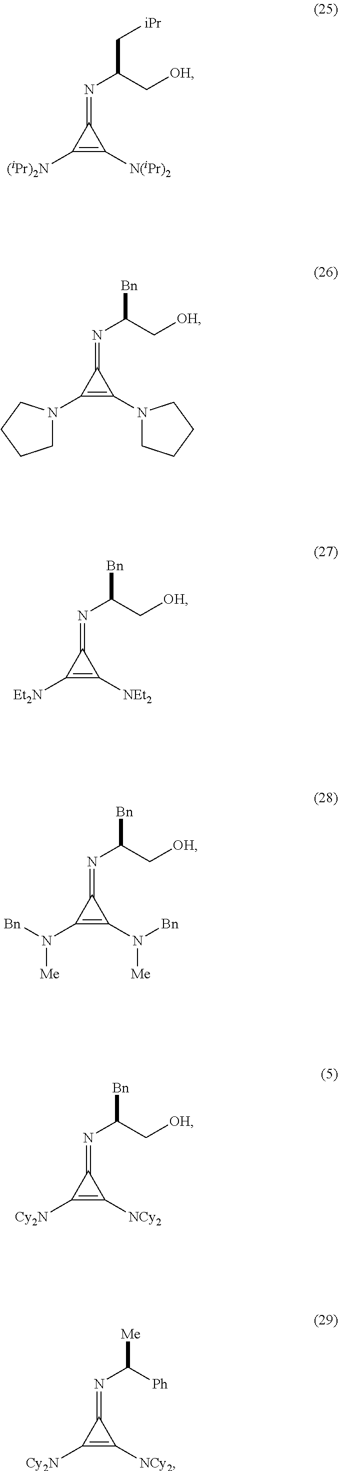 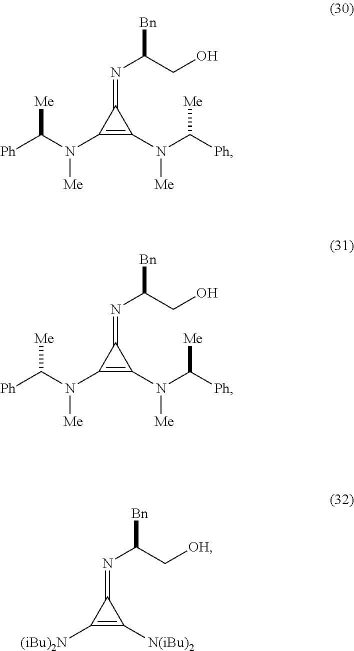
and crystalline forms, hydrates, and salts thereof.

5. The cyclopropenimine Brønsted base catalyst according to claim 1, which is (S)-2-(2,3-bis(dicyclohexylamino)cycloallylamino)-3-phenylpropan-1-ol hydrochloride salt (5.HCl):

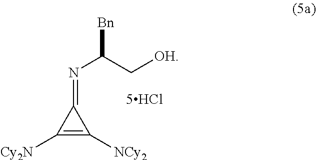

6. A cyclopropenimine scaffold for use as a Brønsted base catalyst, which forms a cyclopropenium ion upon protonation of an imino nitrogen, the scaffold having the structure: 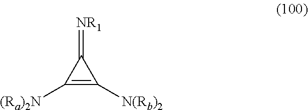
wherein
R₁ is a hydroxyl containing substituent; and
each Rₐ and R_{b} are the same or different and are independently selected from sterically demanding substituents; and
crystalline forms, hydrates, or salts thereof.

7. A process for making a cyclopropenimine suitable for use as a Brønsted base catalyst comprising: contacting a compound of formula (110) with an amine under conditions suitable to form a cyclopropenimine Brønsted base catalyst of formula (100): 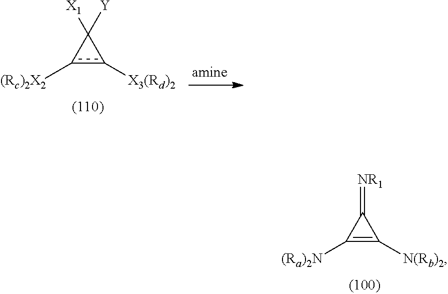
wherein
each X₁₋₃ and Y are the same or different and are independently selected from the group consisting of Cl, N, and no atom;
each Rₐ, R_{b}, R_{c} and R_{d} are the same or different and are independently selected from the group consisting of no atom, amino, aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀cycloalkenyl, C₂₋₁₀alkynyl, halogen, aryloxy, heteroaryloxy, C₂₋₁₀alkoxycarbonyl, C₁₋₁₀alkylthio, C₂₋₁₀alkenylthio, C₂₋₁₀alkynylthio, C₁₋₁₀alkylsulfonyl, C₁₋₁₀alkylsulfinyl aryl-C₁₋₁₀alkyl, heteroaryl-C₁₋₁₀alkyl, aryl-C₁₋₁₀heteroalkyl, heteroaryl-C₁₋₁₀heteroalkyl, a phosphorus group, a silicon group and a boron group, wherein (Rₐ)₂, (R_{b})₂, (R_{c})₂, or (R_{d})₂ are optionally combined to form a 5 to 8-membered carbocyclic or heterocyclic ring; further wherein the aliphatic or aromatic portions of the two substituents are optionally substituted with from 1 to 4 substituents selected from the group consisting of halogen, cyano, nitro, C₁₋₄alkyl, C₂₋₆alkenyl, C₂₋₆alkynyl, aryl, C₁₋₆alkoxy, C₂₋₆alkenyloxy, C₂₋₆alkynyloxy, aryloxy, C₂₋₆alkoxycarbonyl, C₁₋₆ alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus groups; and
amine is one or more amine groups reacted together or sequentially with the compound of formula (110), the amine being selected from the group consisting of a primary, secondary and tertiary amines.

8. The process according to claim 7, wherein the compound of formula (110) is

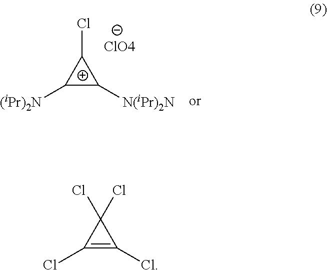

9. The process according to claim 7, wherein the amine is independently selected from the group consisting of tert-Butylamine (^{t}BuNH₂), dicyclohexylamine (HNCy₂), (S)-2-amino-3-phenylpropan-1-ol, and (S)-1-phenylethanamine.

10. A process for making (S)-2-(2,3-bis(dicyclohexylamino)cycloallylamino)-3-phenylpropan-1-ol hydrochloride salt (5.HCl) comprising:
(a) contacting a tetrachlorocyclopropene with dicyclohexylamine for a period of time and under conditions suitable for the tetrachlorocyclopropene and the dicyclohexylamine to react and form a reaction mixture; and
(b) contacting the reaction mixture formed in step (a) with phenylalaninol under conditions suitable to form a (S)-2-(2,3-bis(dicyclohexylamino)cycloallylamino)-3-phenylpropan-1-ol hydrochloride salt (5.HCl).

11. A process for making (S)-2-(2,3-bis(dicyclohexylamino)cycloallylamino)-3-phenylpropan-1-ol comprising:
(a) contacting a tetrachlorocyclopropene with a dicyclohexylamine for a period of time and under conditions suitable for the tetrachlorocyclopropene and the dicyclohexylamine to react and form a reaction mixture;
(b) contacting the reaction mixture formed in step (a) with phenylalaninol under conditions suitable to form (S)-2-(2,3-bis(dicyclohexylamino)cycloallylamino)-3-phenylpropan-1-ol hydrochloride salt (5.HCl);
(c) optionally processing the (S)-2-(2,3-bis(dicyclohexylamino)cycloallylamino)-3-phenylpropan-1-ol hydrochloride salt (5.HCl) formed in step (b) by carrying out one or more of filtering, washing, and drying of the (S)-2-(2,3-bis(dicyclohexylamino)cycloallylamino)-3-phenylpropan-1-ol hydrochloride salt (5.HCl); and
(d) washing the (S)-2-(2,3-bis(dicyclohexylamino)cycloallylamino)-3-phenylpropan-1-ol hydrochloride salt (5.HCl) formed in step (c) with an aqueous organic base.

12. A process for carrying out an organic synthetic reaction comprising:
(a) contacting an organic nucleophile with an organic electrophile in the presence of a cyclopropenimine Brønsted base catalyst under conditions suitable to form a new organic species through the reaction of the organic nucleophile with the organic electrophile, the cyclopropenimine Brønsted base having the structure: 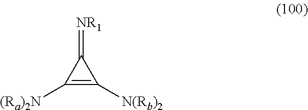
wherein
R₁ is selected from the group consisting of aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀cycloalkenyl, C₂₋₁₀alkynyl, halogen, aryloxy, heteroaryloxy, C₂₋₁₀alkoxycarbonyl, C₁₋₁₀alkylthio, C₂₋₁₀alkenylthio, C₂₋₁₀alkynylthio, C₁₋₁₀alkylsulfonyl, C₁₋₁₀alkylsulfinyl aryl-C₁₋₁₀alkyl, heteroaryl-C₁₋₁₀alkyl, aryl-C₁₋₁₀heteroalkyl, heteroaryl-C₁₋₁₀heteroalkyl; wherein the aliphatic or aromatic portions of R₁ are optionally substituted with from 1 to 4 substituents selected from the group consisting of halogen, cyano, nitro, C₁₋₄-alkyl, C₂₋₆alkenyl, C₂₋₆alkynyl, aryl, C₁₋₆alkoxy, C₂₋₆alkenyloxy, C₂₋₆alkynyloxy, aryloxy, C₂₋₆alkoxycarbonyl, C₁₋₆ alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus groups; and
each Rₐ and R_{b} are the same or different and are independently selected from sterically demanding substituents; and
crystalline forms, hydrates, or salts thereof.

13. The process according to claim 12, wherein the R₁ is substituted with a hydroxy.

14. The process according to claim 12, wherein the organic synthetic reaction is a conjugate addition reaction.

15. The process according to claim 12, wherein the organic synthetic reaction is selected from the group consisting of a Mannich reaction, a Michael reaction, an α-heterofunctionalization of a carbonyl reaction, a Henry (nitroaldol) reaction, an Aza-Henry reaction, a Strecker reaction, and a kinetic resolution reaction, and a desymmetrization reaction.

16. The process according to claim 12, wherein the reaction is a Michael reaction.

17. The process according to claim 16, wherein the Michael reaction is carried out according to the following scheme: 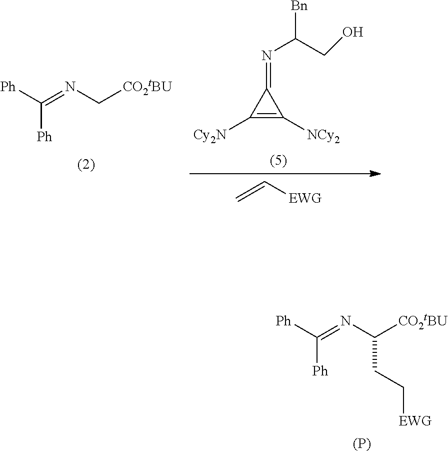
under conditions sufficient to produce a product (P), wherein EWG is an electronic withdrawing group suitable for participating in the reaction.

18. The process according to claim 17, wherein the EWG is selected from the group consisting of:

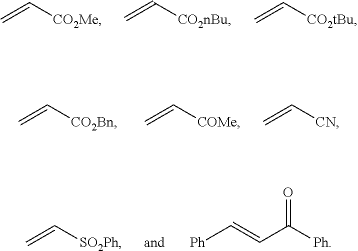

19. The process according to claim 15, wherein the organic synthetic reaction is a Mannich reaction.

20. The process according to claim 19, wherein the Mannich reaction is carried out according to the following scheme: 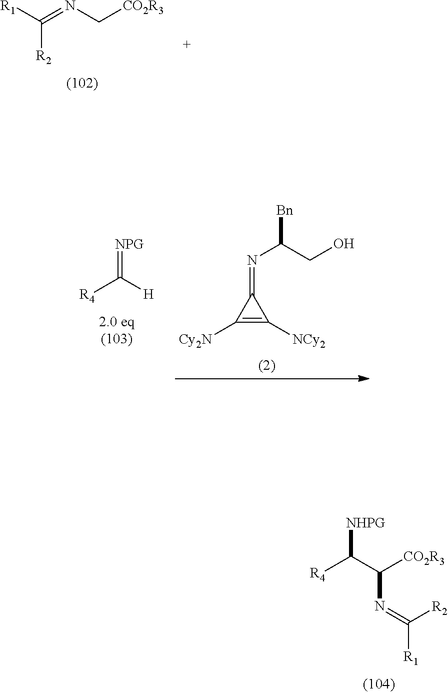
under conditions sufficient to produce a product (104), wherein
R₁, R₂, R₃, R₄ are independently selected from the group consisting of H, C₁₋₆ alkyl, aryl, and heteroaryl; and
PG is a protecting group suitable for participating in the reaction.

21. The process according to claim 20, wherein the PG is tert-butyloxycarbonyl (BOC).

22. The process according to claim 20, wherein R₁ and R₂ are independently selected from the group consisting of H, phenyl, and 4-chlorophenyl.

23. The process according to claim 20, wherein R₃ is selected from the group consisting of tert-butyl, methyl, and benzyl.

24. The process according to claim 20, wherein R₄ is selected from the group consisting of:

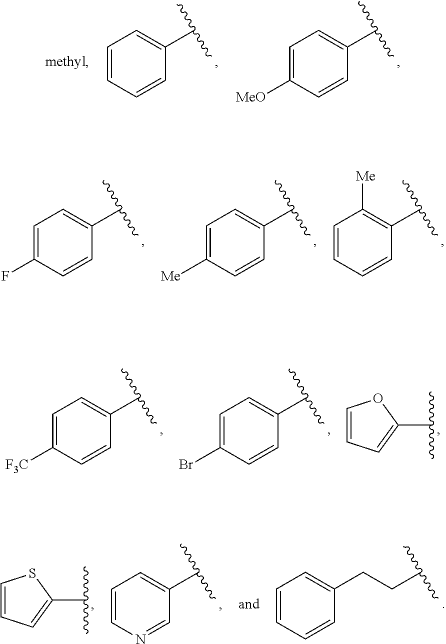

25. The process according to claim 20, wherein product (104) is selected from the group consisting of:

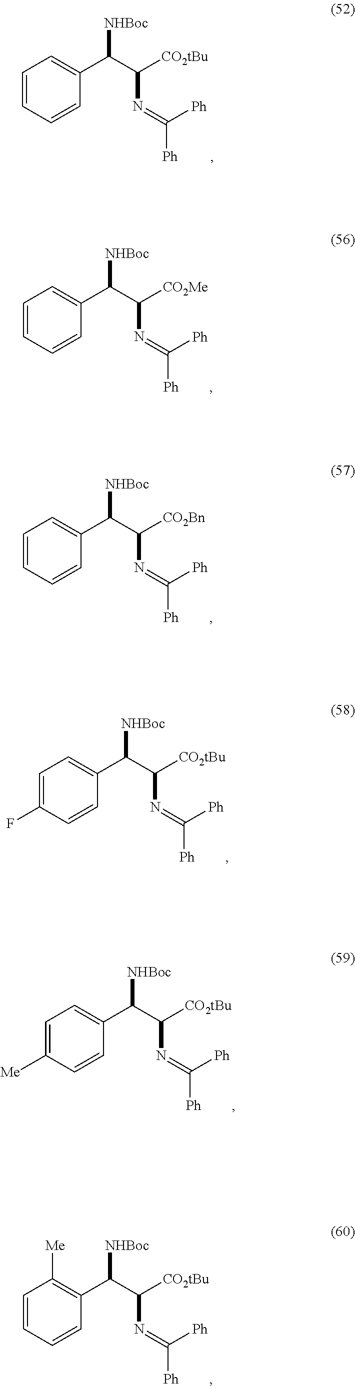 [SMILES: CC(C)(C)OC(=O)N[C@H](c1ccccc1)[C@H](N=C(c1ccccc1)c1ccccc1)C(=O)OC(C)(C)C ; COC(=O)[C@@H](N=C(c1ccccc1)c1ccccc1)[C@H](NC(=O)OC(C)(C)C)c1ccccc1 ; CC(C)(C)OC(=O)N[C@H](c1ccccc1)[C@H](N=C(c1ccccc1)c1ccccc1)C(=O)OCc1ccccc1 ; CC(C)(C)OC(=O)N[C@H](c1ccc(F)cc1)[C@H](N=C(c1ccccc1)c1ccccc1)C(=O)OC(C)(C)C ; Cc1ccc([C@@H](NC(=O)OC(C)(C)C)[C@H](N=C(c2ccccc2)c2ccccc2)C(=O)OC(C)(C)C)cc1 ; Cc1ccccc1[C@@H](NC(=O)OC(C)(C)C)[C@H](N=C(c1ccccc1)c1ccccc1)C(=O)OC(C)(C)C]

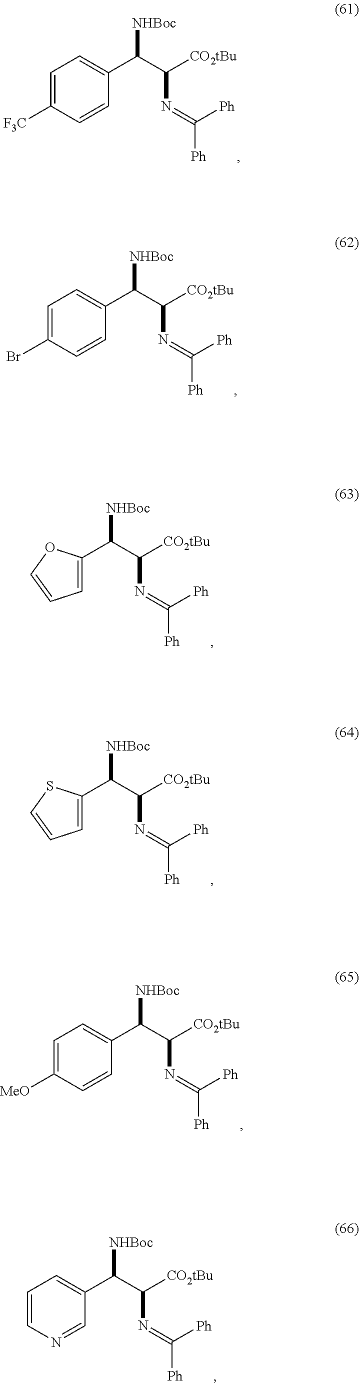 [SMILES: CC(C)(C)OC(=O)N[C@H](c1ccc(C(F)(F)F)cc1)[C@H](N=C(c1ccccc1)c1ccccc1)C(=O)OC(C)(C)C ; CC(C)(C)OC(=O)N[C@H](c1ccc(Br)cc1)[C@H](N=C(c1ccccc1)c1ccccc1)C(=O)OC(C)(C)C ; CC(C)(C)OC(=O)N[C@H](c1ccco1)[C@H](N=C(c1ccccc1)c1ccccc1)C(=O)OC(C)(C)C ; CC(C)(C)OC(=O)N[C@H](c1cccs1)[C@H](N=C(c1ccccc1)c1ccccc1)C(=O)OC(C)(C)C ; COc1ccc([C@@H](NC(=O)OC(C)(C)C)[C@H](N=C(c2ccccc2)c2ccccc2)C(=O)OC(C)(C)C)cc1 ; CC(C)(C)OC(=O)N[C@H](c1cccnc1)[C@H](N=C(c1ccccc1)c1ccccc1)C(=O)OC(C)(C)C]

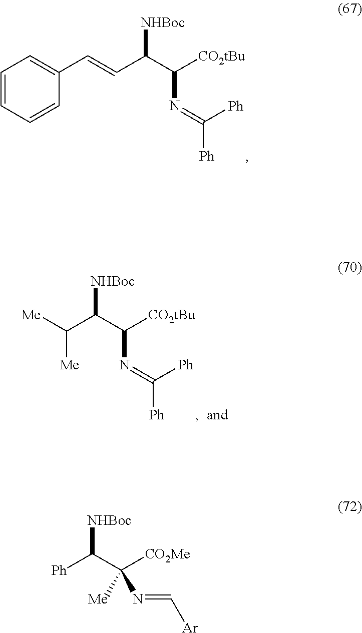

26. The process according to claim 12, wherein the cyclopropenimine Brønsted base catalyst has a structure selected from the group consisting of: 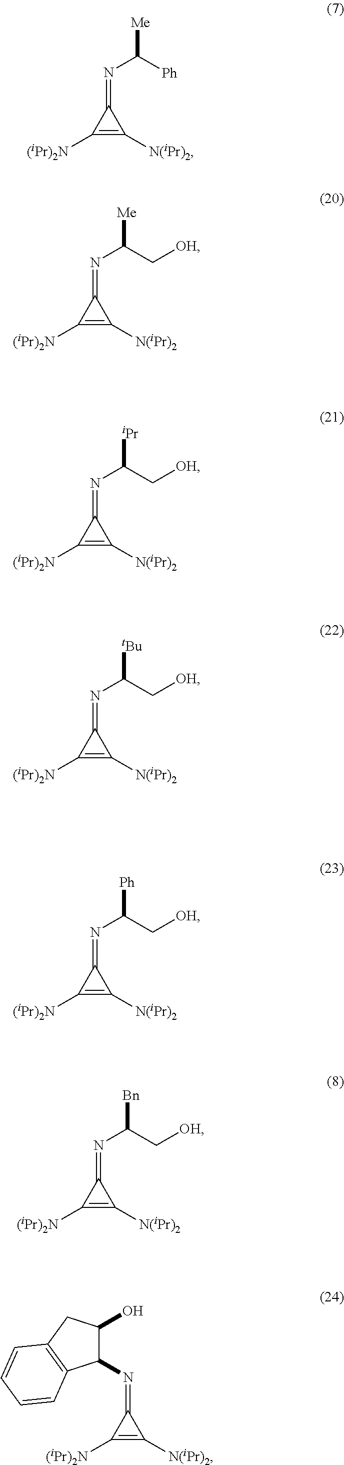 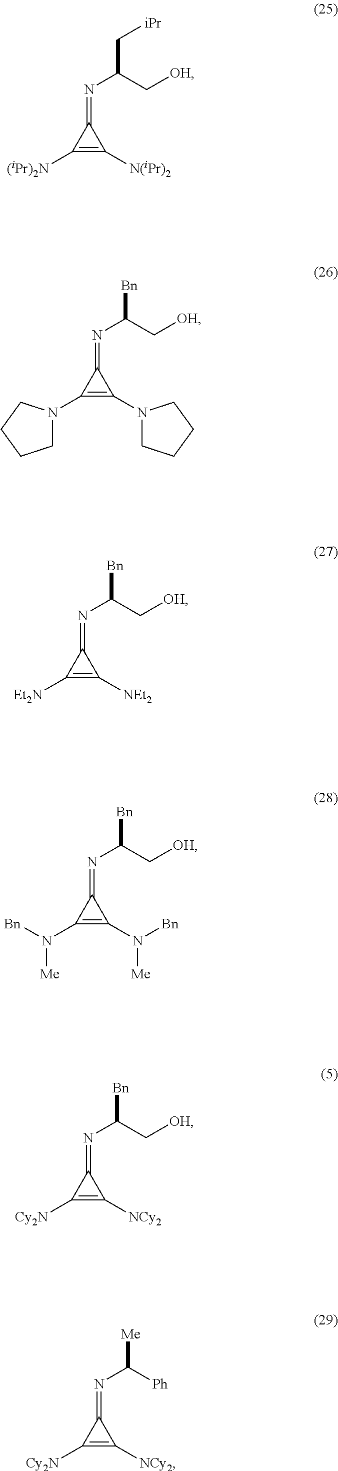 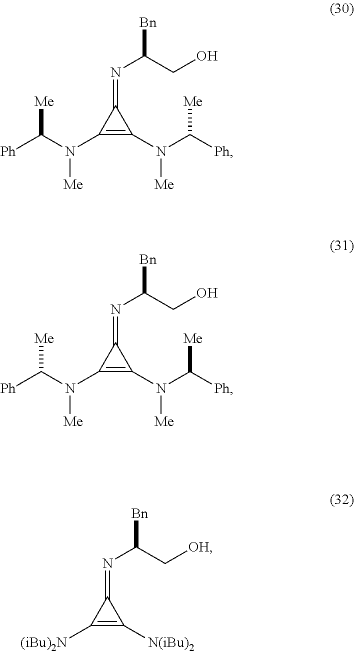
and crystalline forms, hydrates, or salts thereof.

27. The process according to claim 26, wherein the cyclopropenimine Brønsted base catalyst has the structure:

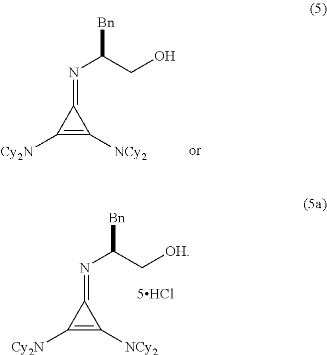

28. A process for catalyzing a proton transfer reaction enantioselectively for the production of an optically enriched organic product, the reaction comprising:
(a) contacting an organic nucleophile with an organic electrophile in the presence of a cyclopropenimine Brønsted base catalyst under conditions suitable to form a new organic species through the reaction of the organic nucleophile with the organic electrophile, the cyclopropenimine Brønsted base having the structure: 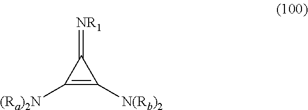
wherein
R₁ is selected from the group consisting of aryl, heteroaryl, C₁₋₁₀alkoxy, C₂₋₁₀alkenyloxy, C₂₋₁₀alkynyloxy, C₁₋₁₀alkyl, C₃₋₁₀cycloalkyl, C₂₋₁₀alkenyl, C₃₋₁₀cycloalkenyl, C₂₋₁₀alkynyl, halogen, aryloxy, heteroaryloxy, C₂₋₁₀alkoxycarbonyl, C₁₋₁₀alkylthio, C₂₋₁₀alkenylthio, C₂₋₁₀alkynylthio, C₁₋₁₀alkylsulfonyl, C₁₋₁₀alkylsulfinyl aryl-C₁₋₁₀alkyl, heteroaryl-C₁₋₁₀alkyl, aryl-C₁₋₁₀heteroalkyl, heteroaryl-C₁₋₁₀heteroalkyl; wherein the aliphatic or aromatic portions of R₁ are optionally substituted with from 1 to 4 substituents selected from the group consisting of halogen, cyano, nitro, C₁₋₄-alkyl, C₂₋₆alkenyl, C₂₋₆alkynyl, aryl, C₁₋₆alkoxy, C₂₋₆alkenyloxy, C₂₋₆alkynyloxy, aryloxy, C₂₋₆alkoxycarbonyl, C₁₋₆ alkylthio, C₁₋₆alkylsulfonyl, C₁₋₆alkylsulfinyl, oxo, imino, thiono, primary amino, carboxyl, C₁₋₆alkylamino, C₁₋₆dialkylamino, amido, nitrogen heterocycles, hydroxy, thiol and phosphorus groups; and
Rₐ and R_{b} are independently selected from sterically demanding substituents; and
crystalline forms, hydrates, or salts thereof.

29. A process for catalyzing a proton transfer reaction enantioselectively for the production of an optically enriched organic product, the reaction comprising: contacting an organic nucleophile with an organic electrophile in the presence of a 2,3-bis(dialkylamino)-cyclopropenimine under conditions suitable to form a new organic species through the reaction of the organic nucleophile with the organic electrophile.

30. The process according to claim 29, wherein the 2,3-bis(dialkylamino)-cyclopropenimine has a structure selected from the group consisting of: 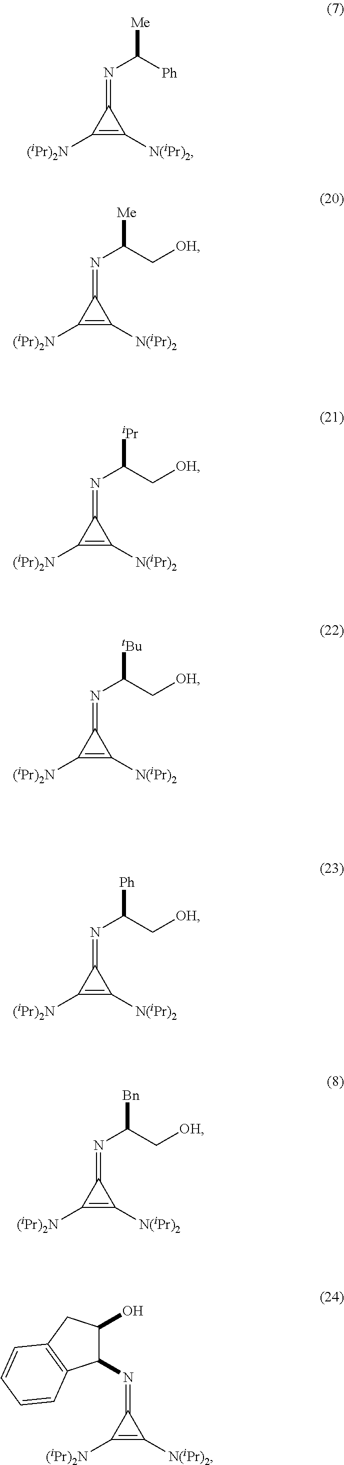 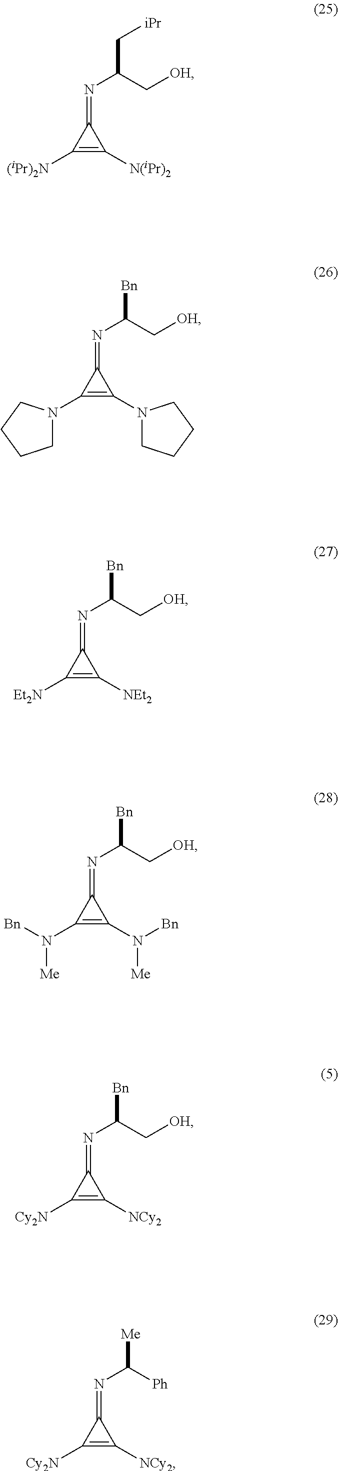 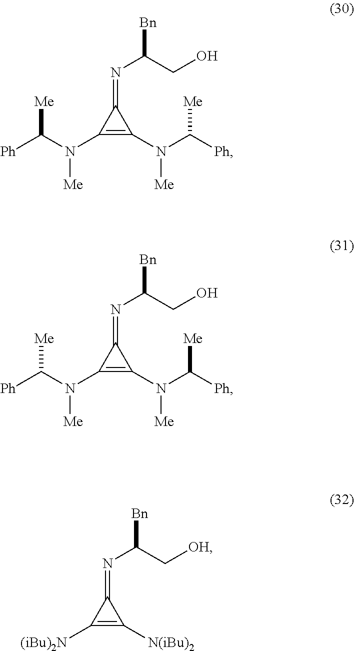
and crystalline forms, hydrates, or salts thereof.

31. The process according to claim 30, wherein the 2,3-bis(dialkylamino)-cyclopropenimine has the structure: [chemical structure]
